SEQUENCE LISTING (30 sequences)

SEQ ID NO: 1 - protein, 1283 aa, Artificial Sequence
  ukcpf1
MSNMQQYDNFINHYAIQKTLRFELQPIGKTREHIQKNGIIEHDEALEQKYQIVKKIIDRFHRKHIDEALSLADFSKDTAMLKRFEELYWKKNKNENEKNEFVKIQSDLRKRVVSFLEGKVEGDARFAKVQQRYGILFDAKIFKDKEFISTACDDIEKDAIEAFKRFATYFTGFHENRKNMYSADEESTAIAYRVINENLPRFLENKARFEKIQHTVDSKTLNEIATELKPVLEKNKLETIFTLNYFQNTLSQAGITYYNTILGGKTKENGEKVQGLNEIINLFNQKNKDTMLPLLKPLYKQILSEEYSTSFTISAFEKDNDVLQAIGSFCNDCIFYAKNNVNGKAYNLLQTVQAFCNSIDTYNDNRLDGLHIERKNLATLSHQVYGEWNILRDALQIHYEAYEQKDNGNNNNYLESKTFSWKALKDALTTYKSLVEEAQDIDENGFIAYFKDMKFKEEIDGKTTSIDLIENIQTRYKSIETILQEDRNNKNNLHQEKEKVATIKGFLDSVKYLQWFLNLMYIASPVDDKDYDFYNELEMYHDTLLPLTTLYNKVRNYMTRKPYSVEKFKLTFEKSTLLDGWDKNKERANLGVILRKGNNYYLGIMNKKYNDIFDSIPGLTTTDYCEKMNYKLLPGPNKMLPKVFFSKKGVQFYKPSQEIIRLYNNKEFKKGDTFNKNSLHKLINFYKESIAKTEDWSVFQFKFKNTNDYADISQFYKDVERQGYKISFDKIDWEYILLLVDEGKLFLFKIYNKDFSPYSKGKPNLHTIYWKNIFSHDNLNNVVYKLNGEAEVFYRKKSIEYPEEILQKGHHVNELKDKFKYPIIKDKRYAEDKFLFHVPITMNFLSKGEPNINQRVQQYIASTSEDYHIIGIDRGERNLLYLSLIDATGKIIKQLSLNTIKNENFNTTIDYHAKLDEKEKKREEARKNWDVIENIKELKEGYLSQVVHQIAKLMVEYKAILVMEDLNTGFKRGRFKVEKQVYQKFEKMMIDKLNYLVLKDRQATQPGGSLKAYQLASSLESFKKLGKQCGMIFYVPAVYTSKIDPTTGFYNFLRVDVSTLNSAHSFFNRFNAIVYNNEQDYFEFHCTYKNFVSEPSLQKNVKSSKMHEYNNLKDTTWVLCSTHHERYKKFKNKSGYFEYKPVNVTQSLKQLFDEAGIDYQAGADLKEAIVTGKNTKLLKGLGEQLNILLAMRYNNGKHGNEEKDYIVSPVKNNYGKFFCTLDGDASLPVDADANGAYAIALKGLMLVERMKSNKDIKGRIDYFISNNEWFNYLIAKNTLNKSK

SEQ ID NO: 2 - DNA, 3852 bp, Artificial Sequence
  ukcpf1
atgagtaacatgcaacagtacgataattttatcaatcattatgcaatacagaaaacactacggtttgaactgcaaccaataggaaaaacacgggagcatattcaaaaaaacgggattattgagcatgatgaagcgcttgagcaaaagtatcagattgttaaaaagattattgatcgttttcacaggaaacacattgatgaagcactttctttagcagatttttcaaaggatactgctatgttaaagcgatttgaggaattatattggaaaaagaacaaaaatgaaaatgaaaaaaatgaatttgtaaaaatacaatctgatttgcgcaaaagggttgtttcttttttagaaggtaaagtagaaggtgatgcaaggtttgcaaaggtacagcaacggtacggtatactatttgatgcaaaaatatttaaagacaaagaatttatatcaacagcctgtgatgatattgaaaaagatgctatagaagcttttaaacgatttgctacctattttacaggtttccatgaaaatcgtaaaaacatgtattccgctgatgaagaatctactgccattgcctatagagtaatcaatgaaaaccttccgcgttttttagaaaataaagcaaggtttgaaaagatacaacacacagttgatagtaaaacgcttaacgagatagcaacagaacttaaaccagtattggaaaagaataagcttgaaaccatatttacgctgaactattttcaaaatacactatcgcaggctggaattacctattataacaccatccttggtggtaaaactaaagaaaatggcgaaaaggttcagggcttaaatgaaattataaatctttttaatcagaaaaataaggatactatgctgccattattaaaaccactgtataagcaaattttaagtgaagagtattccacatcatttaccatttctgcttttgaaaaagataatgatgttttgcaggcaataggttctttttgtaatgattgtattttttatgcaaaaaataatgtcaatggcaaggcatacaaccttttgcaaacggtacaggcattttgtaacagtattgatacctataacgataacaggcttgatggtttgcatatagaaagaaaaaaccttgctaccctttcacatcaggtgtatggcgaatggaatatactgcgggatgcattacagatacactatgaagcgtatgaacaaaaggataatggcaataataataactatctggaaagcaaaaccttttcgtggaaagcattaaaagatgcattaacaacatataaatcactggtggaggaagcacaggatattgatgaaaatggatttatagcttacttcaaggacatgaaatttaaagaagaaattgatggtaaaacaaccagtattgatttaattgaaaatatacaaacccggtataaaagtattgaaaccatattgcaggaagaccgcaacaacaaaaacaatcttcatcaggaaaaggaaaaggtagctacaataaagggctttcttgattcggtaaaatacctgcaatggtttttaaaccttatgtatatagcaagccctgttgatgataaagattatgatttttacaatgagcttgagatgtaccatgacacattgttaccacttactacactgtataataaagtacgcaactatatgacacgcaaaccatattccgttgaaaagtttaaattgacctttgaaaaatcaactcttcttgatggatgggataaaaacaaggaacgagccaatcttggtgttatactaaggaagggtaataattattatcttggcattatgaacaaaaaatacaatgatatatttgatagtatccccggacttactaccactgactattgtgaaaagatgaactataaattgctgcctggccccaataaaatgttgcctaaagtatttttttcaaaaaaaggtgtacagttttacaaaccttcacaggaaatcatccgtttgtataataataaggaatttaaaaagggagatacctttaataaaaatagtttgcacaaacttattaatttttataaagaatccattgcaaaaactgaagattggagtgtattccagtttaaatttaaaaacacaaacgattatgccgatataagccagttttataaagatgttgaaagacagggctataaaattagttttgataagattgattgggaatatatactattgctggtagatgaaggaaaattatttcttttcaagatttacaataaggacttttcaccctatagcaagggtaagcctaacctgcataccatttactggaaaaatattttttcccatgataatttaaacaatgttgtgtataagcttaatggtgaagccgaagttttttaccggaagaaatccatagagtatccagaagaaatactacaaaaaggtcatcatgtcaatgagttaaaggataaatttaagtaccctattattaaagataaacgctatgccgaagacaaatttcttttccacgttcccattaccatgaactttttatcaaaaggggaacctaatataaatcaaagggttcagcaatatattgcatctactagtgaagattatcatattattggtattgaccggggtgaaagaaatttactgtatctgtcgctcatagatgcaacaggcaaaataataaaacaactatcgctcaataccattaaaaatgaaaattttaacacaacaatagattaccatgcaaaattagatgaaaaagaaaagaaaagagaagaagcaagaaaaaattgggatgtcatagaaaatattaaagagcttaaagaaggatacctgtcacaggttgttcatcagattgcaaaacttatggttgaatataaagcaatcctggtgatggaggatttgaataccggttttaagcgtggaaggtttaaagtggaaaaacaggtatatcaaaaatttgaaaaaatgatgatagataaattaaattaccttgtgttgaaggacaggcaggctacacagccaggtggaagcttaaaagcatatcaacttgcatcgtcattggaatcgtttaaaaaacttggtaaacagtgtggcatgattttttatgttcctgcagtgtatacaagcaagattgatccaacaactggtttttacaactttttaagggtagatgtttcaacattgaacagcgcgcacagtttttttaacaggtttaatgcaattgtttataataatgagcaggattattttgaatttcattgcacctacaaaaattttgtttctgaaccgtcattgcaaaaaaatgtaaaaagcagcaaaatgcacgagtataacaatttaaaagatactacatgggtactatgctctactcatcatgagcgctataaaaagtttaaaaataaaagcggttattttgagtataaaccagttaatgtaacccaaagcttaaagcagctttttgacgaggctggtattgattatcaagcaggggctgacttaaaggaagcgatagtaaccggtaaaaatactaaactattaaagggtctgggcgagcagttgaacatactattagccatgcgatataacaatggtaaacatgggaatgaagaaaaagattatattgtttctccagtaaaaaataattatggaaaatttttctgcacgcttgatggtgatgcatcattacctgttgacgccgatgccaatggtgcgtatgccatagcattgaaagggcttatgcttgttgaaaggatgaaaagcaataaagatataaaagggaggatagattattttatttcaaataatgagtggtttaactatttaattgcaaagaacacattaaataaatcaaaataa

SEQ ID NO: 3 - RNA, 18 nt, Artificial Sequence
  gRNA-DR
auuucuacuauuguagau

SEQ ID NO: 4 - DNA, 23 bp, Artificial Sequence
  gTGW6-1
actacaaaaccggcaacctgtac

SEQ ID NO: 5 - DNA, 23 bp, Artificial Sequence
  gTGW6-2
tttcaccgacagcagcatgaact

SEQ ID NO: 6 - DNA, 23 bp, Artificial Sequence
  gTGW6-3
ttgacctgccaggctatcctgat

SEQ ID NO: 7 - DNA, 23 bp, Artificial Sequence
  gTGW6-4
ggtccggatagtcacttggttgc

SEQ ID NO: 8 - DNA, 23 bp, Artificial Sequence
  gTGW6-5
cgtgtagctggggctgtacgtgt

SEQ ID NO: 9 - DNA, 52 bp, Artificial Sequence
  N-B-i3g1-ssDNA0
cgacattccgaagaacgctgaagcgctgggggcaaattgtgcaatttgcggc

SEQ ID NO: 10 - RNA, 43 nt, Artificial Sequence
  gRNA-trans
agagaaugugugcauagucacaccccccagcgcuucagcguuc

SEQ ID NO: 11 - protein, 7 aa, Artificial Sequence
  Synthetic sequence
PKKKRKV

SEQ ID NO: 12 - protein, 20 aa, Artificial Sequence
  synthetic sequence
AVKRPAATKKAGQAKKKKLD

SEQ ID NO: 13 - protein, 9 aa, Artificial Sequence
  synthetic sequence
PAAKRVKLD

SEQ ID NO: 14 - protein, 25 aa, Artificial Sequence
  synthetic sequence
MSRRRKANPTKLSENAKKLAKEVEN

SEQ ID NO: 15 - protein, 9 aa, Artificial Sequence
  synthetic sequence
KLKIKRPVK

SEQ ID NO: 16 - protein, 5 aa, Artificial sequence
  synthetic sequence
KIPIK

SEQ ID NO: 17 - protein, 4 aa, Artificial Sequence
  YFFP gene
YFFP

SEQ ID NO: 18 - DNA, 141 bp, Artificial Sequence
  repeat-spacer-repeat DR-Sp-DR
ttgacagctagctcagtcctaggtataatactagtgtctaaaggtattataaaatttctactattgtagatagagcgcaattaattattgcggatattcgtctaaaggtattataaaatttctactattgtagattttttt

SEQ ID NO: 19 - DNA, 44 bp, Artificial Sequence
  pAtUBQ-F-UnCpf1 primer
gagagagacgaaacacaaaccatggactacaaggaccacgacgg

SEQ ID NO: 20 - DNA, 51 bp, Artificial Sequence
  UnCpf1-R-tUBQ primer
ttcttgataagagtctcttaggatcctcactccaccttgcgcttcttcttg

SEQ ID NO: 21 - DNA, 27 bp, Artificial Sequence
  AsDR-EBE-S1 primer
agattctcttagggataacagggtaat

SEQ ID NO: 22 - DNA, 27 bp, Artificial Sequence
  AsDR-EBE-A1T primer
aaaaattaccctgttatccctaagaga

SEQ ID NO: 23 - DNA, 27 bp, Artificial Sequence
  AsDR-EBE-S2 primer
agattctctattaccctgttatcccta

SEQ ID NO: 24 - DNA, 27 bp, Artificial Sequence
  ASDR-EBE-A2T primer
aaaatagggataacagggtaatagaga

SEQ ID NO: 25 - DNA, 27 bp, Artificial Sequence
  AsDR-TT4-S269 primer
agatctattcacaggcgacaagtcgac

SEQ ID NO: 26 - DNA, 27 bp, Artificial Sequence
  AsDR-TT4-A269T primer
aaaagtcgacttgtcgcctgtgaatag

SEQ ID NO: 27 - DNA, 18 bp, Artificial Sequence
  AtPDS-F primer
ggtcctttgcaggtatct

SEQ ID NO: 28 - DNA, 21 bp, Artificial Sequence
  AtPDS-R primer
ttcaaaggcttagcaggacga

SEQ ID NO: 29 - DNA, 20 bp, Artificial Sequence
  DNMT1-F primer
cgggaaccaagcaagaagtg

SEQ ID NO: 30 - DNA, 20 bp, Artificial Sequence
  DNMT1-R primer
gggcaacacagtgagactcc

(19) United States
(12) Patent Application Publication
(10) Pub. No.: US 2022/0186206 A1
(43) Pub. Date: Jun. 16, 2022

(54) NOVEL Cas ENZYME AND SYSTEM, AND USE THEREOF

(71) Applicant: SHANDONG SHUNFENG BIOTECHNOLOGY CO., LTD., Jinan (CN)

(72) Inventor: Yafeng LIANG, Jinan (CN)

(73) Assignee: SHANDONG SHUNFENG BIOTECHNOLOGY CO., LTD., Jinan (CN)

(21) Appl. No.: 17/648,299

(22) Filed: Jan. 18, 2022

Related U.S. Application Data
(63) Continuation of application No. PCT/CN2021/129034, filed on Nov. 5, 2021.

(30) Foreign Application Priority Data
Nov. 11, 2020 (CN) 202011255433.3
Nov. 4, 2021 (CN) 202111298497.6

(51) Int. Cl. C12N 15/10; C12N 9/22; C12N 15/63; C12N 15/113

(52) U.S. Cl. CPC C12N 15/102; C12N 9/22; C12N 2310/20; C12N 15/113; C12N 15/63

(57) ABSTRACT

A CRISPR-associated (Cas) protein, a fusion protein including the Cas protein, and a nucleic acid encoding either of the proteins are provided. The Cas protein is any one from the group consisting of a Cas protein having an amino acid sequence with at least 95% sequence identity with SEQ ID NO: 1 and basically retaining a biological function of SEQ ID NO: 1; a Cas protein having an amino acid sequence obtained through a substitution, a deletion, or an addition of one or more amino acids based on SEQ ID NO: 1 and basically retaining the biological function of SEQ ID NO: 1; and a Cas protein comprising an amino acid sequence shown in SEQ ID NO: 1.

### CROSS REFERENCE TO THE RELATED APPLICATIONS

This is a continuation application of the national phase entry of International Application No. PCT/CN2021/129034, filed on Nov. 5, 2021, which is based upon and claims priority to Chinese Patent Application No. 202011255433.3, filed on Nov. 11, 2020, and Chinese Patent Application No. 202111298497.6, filed on Nov. 4, 2021, the entire contents of which are incorporated herein by reference.

### SEQUENCE LISTING

The instant application contains a Sequence Listing which has been submitted in ASCII format via EFS-Web and is hereby incorporated by reference in its entirety. Said ASCII copy is named GBSDSF005-PKG_SL.txt, created on Jan. 4, 2022, and is 22,191 bytes in size.

### TECHNICAL FIELD

The present disclosure relates to the field of gene editing, and in particular to the technical field of clustered regularly interspaced short palindromic repeat (CRISPR). Specifically, the present disclosure relates to a novel CRISPR-associated (Cas) effector protein, a fusion protein including the Cas effector protein, and a nucleic acid encoding either of the proteins.

### BACKGROUND

The CRISPR/Cas technology is a widely-used gene editing technology, where RNA guidance is used specifically to bind to a target sequence on a genome and cleave DNA to produce double strand breaks (DSBs), and site-directed gene editing is conducted through biological non-homologous end joining (NHEJ) or homologous recombination.

The CRISPR/Cas9 system is the most common type II CRISPR system, which recognizes protospacer adjacent motif (PAM) of 3′-NGG and cleaves a target sequence to produce blunt-ended fragments. The CRISPR/Cas Type V system is a newly discovered CRISPR system, such as Cpf1, C2c1, CasX, and CasY. However, the different CRISPR/Cas systems existing currently have different advantages and disadvantages. For example, Cas9, C2c1, and CasX all require two RNAs for RNA guidance, while Cpf1 only requires one guide RNA (gRNA) and can be used for multiplex gene editing. CasX has a size of 980 amino acids, while common Cas9, C2c1, CasY, and Cpf1 usually have a size of about 1,300 amino acids.

Given that the currently available CRISPR/Cas systems are limited by some shortcomings, it is of great significance for the development of biotechnology to develop a robust novel CRISPR/Cas system with prominent performance in many aspects.

### SUMMARY

The inventors of the present disclosure discover a novel endonuclease (Cas enzyme) through many experiments of trial and error. On the basis of this discovery, the inventors develop a novel CRISPR/Cas system, and a gene editing method and nucleic acid detection method based on the system.

Cas Effector Protein

In an aspect, the present disclosure provides a Cas protein, where the Cas protein is an effector protein in a CRISPR/Cas system, and bioinformatics analysis shows that the Cas protein is a protein of the Cas12a (Cpf1) family.

In an embodiment, an amino acid sequence of the Cas protein may have at least 80%, at least 85%, at least 90%, at least 91%, at least 92%, at least 93%, at least 94%, at least 95%, at least 96%, at least 97%, at least 98%, or at least 99% sequence identity with SEQ ID NO: 1, and may basically retain a biological function of SEQ ID NO: 1.

In an embodiment, the amino acid sequence of the Cas protein may be obtained through substitution, deletion, or addition of one or more amino acids (for example, substitution, deletion, or addition of 1, 2, 3, 4, 5, 6, 7, 8, 9, or 10 amino acids) based on a sequence shown in SEQ ID NO: 1.

In an embodiment, the Cas protein may include the amino acid sequence shown in SEQ ID NO: 1.

In an embodiment, the Cas protein may be the amino acid sequence shown in SEQ ID NO: 1.

In an embodiment, the Cas protein may be a derivatized protein with the same biological function as a protein of the sequence shown in SEQ ID NO: 1.

The biological function includes, but is not limited to, activity to bind to gRNA, endonuclease activity, and activity to bind to a specific site of a target sequence and cleave the target sequence under guidance of gRNA (including but not limited to Cis cleavage activity and Trans cleavage active).

The present disclosure also provides a fusion protein including the Cas protein described above and a modification part.

In an embodiment, the modification part may be another protein or polypeptide, a detectable label, or any combination thereof.

In an embodiment, the modification part may be selected from the group consisting of an epitope tag, a reporter gene sequence, a nuclear localization signal (NLS) sequence, a targeting moiety, a transcriptional activation domain (such as VP64), a transcriptional repression domain (such as KRAB or SID domain), a nuclease domain (such as Fok1), and a domain with activity selected from the group consisting of nucleotide deaminase activity, methylase activity, demethylase activity, transcription activation activity, transcription repression activity, transcription release factor activity, histone modification activity, nuclease activity, single-stranded RNA cleavage activity, double-stranded RNA cleavage activity, single-stranded DNA cleavage activity, double-stranded DNA cleavage activity, and nucleic acid binding activity; and any combination thereof. The NLS sequence is well known to those skilled in the art, and examples thereof include, but are not limited to, SV40 large T antigen, EGL-13, c-Myc, and TUS protein.

In an embodiment, the NLS sequence may be located at, close to, or proximate to a terminus (such as N-terminus, C-terminus, or both termini) of the Cas protein of the present disclosure.

The epitope tag is well known to those skilled in the art, including but not limited to His, V5, FLAG, HA, Myc, VSV-G, and Trx; and those skilled in the art can select other suitable epitope tags (such as purification, detection, or tracing tag).

The reporter gene sequence is well known to those skilled in the art, and examples thereof include but are not limited to GST, HRP, CAT, GFP, HcRed, DsRed, CFP, YFP, and BFP.

In an embodiment, the fusion protein of the present disclosure may include a domain capable of binding to a DNA molecule or an intracellular molecule, such as maltose-binding protein (MBP), DNA binding domain (DBD) of Lex A, and DBD of GAL4.

In an embodiment, the fusion protein of the present disclosure may include a detectable label, such as a fluorescent dye, such as fluorescein isothiocyanate (FITC) or 4′,6-diamidino-2-phenylindole (DAPI).

In an embodiment, the Cas protein of the present disclosure may be optionally coupled to, conjugated with, or fused to the modification part through a linker.

In an embodiment, the modification part may be directly linked to the N-terminus or C-terminus of the Cas protein of the present disclosure.

In an embodiment, the modification part may be linked to the N-terminus or C-terminus of the Cas protein of the present disclosure through a linker. Such linkers are well known in the art, and examples thereof include, but are not limited to, a linker with one or more (such as 1, 2, 3, 4, or 5) amino acids (such as Glu or Ser) or amino acid derivatives (such as Ahx, β-Ala, GABA, or Ava), or a polyethylene glycol (PEG) linker.

A production method of the Cas protein, protein derivative, or fusion protein of the present disclosure is not limited. For example, the Cas protein, protein derivative, or fusion protein can be produced by a genetic engineering method (recombinant technology), or can be produced by a chemical synthesis method.

One or more amino acid residues of the Cas protein shown in SEQ ID NO: 1 of the present disclosure can be modified. The modification may involve mutation of one or more amino acid residues of the Cas protein. The one or more mutations may be in one or more catalytically-active domains of the Cas protein, and the above-mentioned mutations will cause the nuclease activity of the Cas protein to decrease or disappear. In an embodiment, the one or more mutations may include 1, 2, or 3 mutations. In an embodiment, the mutation may be D873A, E964A, or D1232A encoded with reference to amino acid positions of SEQ ID NO: 1.

In an embodiment, the Cas protein of the present disclosure may have one or more catalytic sites selected from the group consisting of D873, E964, and D1232 of the sequence shown in SEQ ID NO: 1. In an embodiment, the Cas protein of the present disclosure may have all of the above catalytic sites (D873, E964, and D1232 of the sequence shown in SEQ ID NO: 1).

The gRNA of the Cas protein of the present disclosure may include a guide sequence to hybridize with a target sequence, where the target sequence is located at the 3′ terminus of PAM; and the PAM sequence is 5′-YYV-3′, where Y=C/T and V=C/G/A.

It is clear to those skilled in the art that a structure of a protein can be changed without adversely affecting the activity and functionality of the protein. For example, one or more conservative amino acid substitutions can be introduced into an amino acid sequence of a protein without adversely affecting the activity and/or three-dimensional (3D) structure of the protein molecule. Those skilled in the art are aware of examples and implementations of the conservative amino acid substitutions. Specifically, an amino acid residue can be substituted by another amino acid residue that belongs to the same group as the amino acid residue to be substituted. That is, a nonpolar amino acid residue can be substituted by another nonpolar amino acid residue; an uncharged polar amino acid residue can be substituted by another uncharged polar amino acid residue; a basic amino acid residue can be substituted by another basic amino acid residue; and an acidic amino acid residue can be substituted by another acidic amino acid residue. Such substituted amino acid residues may be or may not be encoded by genetic codes. As long as a substitution does not result in the loss of biological activity of a protein, a conservative substitution in which an amino acid is substituted by another amino acid belonging to the same group falls within the scope of the present disclosure. Therefore, the protein of the present disclosure may include one or more conservative substitutions in the amino acid sequence, and these conservative substitutions may be preferably generated according to Table 1. In addition, the present disclosure also covers proteins with one or more other non-conservative substitutions, as long as the non-conservative substitutions do not significantly affect the desired function and biological activity of the protein of the present disclosure.

Conservative amino acid substitutions can be made at one or more predicted non-essential amino acid residues. Non-essential amino acid residues are amino acid residues that can be changed (deleted or substituted) without changing the biological activity, while essential amino acid residues are required for biological activity. A conservative amino acid substitution refers to a substitution in which an amino acid residue is substituted by an amino acid residue with a similar side chain. An amino acid substitution can be made in a non-conservative region of the Cas protein described above. Generally, such a substitution is not made to a conservative amino acid residue or an amino acid residue located within a conservative motif, because such a residue is required for protein activity. However, those skilled in the art should understand that a functional variant may have few conservative or non-conservative variations in conservative regions.

TABLE 1

| Initial residue | Representative substitution | Preferred substitution |
|---|---|---|
| Ala (A) | Val; Leu; Ile | Val |
| Arg (R) | Lys; Gln; Asn | Lys |
| Asn (N) | Gln; His; Lys; Arg | Gln |
| Asp (D) | Glu | Glu |
| Cys (C) | Ser | Ser |
| Gln (Q) | Asn | Asn |
| Glu (E) | Asp | Asp |
| Gly (G) | Pro; Ala | Ala |
| His (H) | Asn; Gln; Lys; Arg | Arg |
| Ile (I) | Leu; Val; Met; Ala; Phe | Leu |
| Leu (L) | Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Leu |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala | Leu |

It is well known in the art that one or more amino acid residues can be changed (substituted, deleted, truncated, or inserted) at the N-terminus and/or C-terminus of a protein while still retaining the functional activity of the protein. Therefore, a protein obtained by changing one or more amino acid residues at the N-terminus and/or C-terminus of the Cas protein while retaining its desired functional activity is also within the scope of the present disclosure. The change may include a change introduced by a modern molecular method such as PCR, and the method includes PCR amplification that alters or extends a protein coding sequence by introducing an amino acid coding sequence into an oligonucleotide used in PCR amplification.

It should be recognized that a protein can be altered in various ways, including amino acid substitution, deletion, truncation, and insertion, and methods for such operations are generally known in the art. For example, amino acid sequence variants of the protein described above can be prepared through mutation of DNA. It can also be completed through other mutagenesis forms and/or through directed evolution, for example, known mutagenesis, recombination, and/or shuffling methods can be used in combination with a related screening method to achieve the substitution, deletion, and/or insertion of one or more amino acids.

Those skilled in the art can understand that these small amino acid changes in the Cas protein of the present disclosure can be naturally present (for example, natural mutations) or can be induced (for example, using r-DNA technology), which do not affect the function or activity of the protein. If these mutations occur in a catalytic domain, an active site, or another functional domain of the protein, the properties of the polypeptide may be changed, but the polypeptide may retain its activity. If existing mutations are not close to a catalytic domain, an active site, or another functional domain, it can be expected that there is a small impact.

Those skilled in the art can identify essential amino acids of the Cas protein of the present disclosure according to a method known in the art, such as site-directed mutagenesis or protein evolution or bioinformatics analysis. The catalytic domain, active site, or another functional domain of the protein can also be determined by physical analysis of the structure, for example, it can be determined through a technique such as nuclear magnetic resonance (NMR), crystallography, electron diffraction, or photoaffinity labeling in combination with a putative amino acid mutation at a key position.

Nucleic Acid of Cas Protein

In another aspect, the present disclosure provides an isolated polynucleotide, including:

(a) a polynucleotide sequence encoding the Cas protein or fusion protein of the present disclosure;

(b) a polynucleotide with a sequence shown in SEQ ID NO: 2;

(c) a sequence obtained through substitution, deletion, or addition of one or more bases (such as substitution, deletion, or addition of 1, 2, 3, 4, 5, 6, 7, 8, 9, or 10 bases) based on a sequence shown in SEQ ID NO: 2;

(d) a polynucleotide that has a sequence homology ≥80% (preferably ≥90%, more preferably ≥95%, and most preferably ≥98%) with the sequence shown in SEQ ID NO: 2 and encodes the polypeptide shown in SEQ ID NO: 1; or,

(e) a polynucleotide complementary to any one selected from the group consisting of the polynucleotides described in (a) to (d).

In an embodiment, the nucleotide sequence described in any one of (a) to (e) may be codon-optimized for expression in a prokaryotic cell. In an embodiment, the nucleotide sequence described in any one of (a) to (e) may be codon-optimized for expression in a eukaryotic cell.

In an embodiment, the cell may be an animal cell, such as a mammalian cell.

In an embodiment, the cell may be a human cell.

In an embodiment, the cell may be a plant cell, such as a cell possessed by a cultivated plant (such as cassava, corn, sorghum, wheat, or rice), algae, a tree, or a vegetable.

In an embodiment, the polynucleotide may preferably be single-stranded or double-stranded.

Direct Repeat

In another aspect, the present disclosure provides an engineered direct repeat that forms a complex with the Cas protein described above.

The direct repeat can be linked to a guide sequence capable of hybridizing with the target sequence to form a gRNA.

The hybridization of the target sequence with the gRNA means that the target sequence and the nucleic acid sequence of gRNA have at least 70%, 75%, 80%, 85%, 90%, 91%, 92%, 93%, 94%, 95%, 96%, 97%, 98%, 99%, or 100% identity, and thus can be hybridized to form a complex; or means that at least 12, 15, 16, 17, 18, 19, 20, 21, 22, or more bases in the target sequence are complementary to and paired with that in the nucleic acid sequence of gRNA to form a complex.

In some embodiments, the direct repeat may have at least 90% sequence identity with SEQ ID NO: 3. In some embodiments, the direct repeat may have a sequence obtained through substitution, deletion, or addition of one or more bases (such as substitution, deletion, or addition of 1, 2, 3, 4, 5, 6, 7, 8, 9, or 10 bases) based on the sequence shown in SEQ ID NO: 3.

In some embodiments, the direct repeat may have a sequence shown in SEQ ID NO: 3.

gRNA

In another aspect, the present disclosure provides a gRNA, including a first segment and a second segment. The first segment is also called “framework region”, “protein binding segment”, “protein binding sequence”, or “direct repeat”; and the second segment is also called “nucleic acid-targeted targeting sequence”, “nucleic acid-targeted targeting segment”, or “target sequence-targeted guide sequence”.

The first segment of the gRNA can interact with the Cas protein of the present disclosure, such that the Cas protein and the gRNA form a complex.

The nucleic acid-targeted targeting sequence or the nucleic acid-targeted targeting segment of the present disclosure may include a nucleotide sequence complementary to a sequence in the target nucleic acid. In other words, the nucleic acid-targeted targeting sequence or the nucleic acid-targeted targeting segment of the present disclosure can interact with the target nucleic acid in a sequence-specific manner through hybridization (namely, base pairing). Therefore, the nucleic acid-targeted targeting sequence or the nucleic acid-targeted targeting segment can be changed, or can be modified to hybridize with any desired sequence in the target nucleic acid. The nucleic acid may be selected from the group consisting of DNA and RNA.

The nucleic acid-targeted targeting sequence or the nucleic acid-targeted targeting segment may have at least 60% (such as at least 65%, at least 70%, at least 75%, at least 80%, at least 85%, at least 90%, at least 95%, at least 97%, at least 98%, at least 99%, or 100%) complementarity with a target sequence of the target nucleic acid.

The “framework region”, “protein binding segment”, “protein binding sequence”, or “direct repeat” of gRNA of the present disclosure can interact with the CRISPR protein (or Cas protein). The gRNA of the present disclosure guides the Cas protein interacting therewith to a specific nucleotide sequence in the target nucleic acid under the action of the nucleic acid-targeted targeting sequence.

Preferably, the gRNA may include a first segment and a second segment in a direction from 5′ terminus to 3′ terminus.

In the present disclosure, the second segment can also be understood as a guide sequence to hybridize with the target sequence.

The gRNA of the present disclosure can form a complex with the Cas protein.

Vector

The present disclosure also provides a vector, including the Cas protein, the isolated nucleic acid, or the polynucleotide described above. Preferably, the vector may also include a regulatory element operably linked to the Cas protein, the isolated nucleic acid, or the polynucleotide.

In an embodiment, the regulatory element may be one or more selected from the group consisting of an enhancer, a transposon, a promoter, a terminator, a leader sequence, a polyadenylate sequence, and a marker gene.

In an embodiment, the vector may include a cloning vector, an expression vector, a shuttle vector, and an integration vector.

In some embodiments, a vector included in the system may be a viral vector (such as a retroviral vector, a lentiviral vector, an adenoviral vector, an adeno-associated virus (AAV) vector, or a herpes simplex virus (HSV) vector), or may be a plasmid, a virus, a cosmid, or a phage, which are well known to those skilled in the art.

Vector System

The present disclosure provides an engineered non-natural vector system or a CRISPR-Cas system, which includes the Cas protein or a nucleic acid sequence encoding the Cas protein, and a nucleic acid encoding one or more gRNAs.

In an embodiment, the nucleic acid sequence encoding the Cas protein and the nucleic acid encoding one or more gRNAs may be artificially synthesized.

In an embodiment, the nucleic acid sequence encoding the Cas protein and the nucleic acid encoding one or more gRNAs do not co-exist naturally.

The one or more gRNAs target one or more target sequences in the cell. The one or more target sequences hybridize with a genomic locus of a DNA molecule encoding one or more gene products, and guide the Cas protein to the genomic locus of the DNA molecule encoding the one or more gene products; and after the Cas protein reaches the position of the target sequence, the target sequence is modified, edited, or cleaved, such that the expression of the one or more gene products is changed or modified.

The cell of the present disclosure may include one or more selected from the group consisting of an animal cell, a plant cell, and a microorganism.

In some embodiments, the Cas protein may be codon-optimized for expression in a cell.

In some embodiments, the Cas protein may guide the cleavage of one or two strands at the position of the target sequence.

The present disclosure also provides an engineered non-natural vector system, including one or more vectors; and the one or more vectors include:

a) a first regulatory element operably linked to the gRNA and

b) a second regulatory element operably linked to the Cas protein;

where the components (a) and (b) are located on the same vector or different vectors of the system.

The first and second regulatory elements may include a promoter (such as a constitutive promoter or an inducible promoter), an enhancer (such as a 35S promoter or a 35S enhanced promoter), an internal ribosome entry site (IRES), and other expression control elements (such as a transcriptional termination signal, such as a polyadenylation signal and a poly-U sequence).

In some embodiments, a vector in the system may be a viral vector (such as a retroviral vector, a lentiviral vector, an adenoviral vector, an AAV vector, or an HSV vector), or may be a plasmid, a virus, a cosmid, or a phage, which are well known to those skilled in the art.

In some embodiments, the system provided herein may be in a delivery system. In some embodiments, the delivery system may be a nanoparticle, a liposome, an exosome, a microvesicle, or a gene gun.

In an embodiment, when the target sequence is DNA, the target sequence may be located at the 3′-terminus of PAM, and the PAM may have a sequence shown in 5′-YYV-3′, where Y=C/T and V=C/G/A.

In an embodiment, the target sequence may be a DNA or RNA sequence derived from a prokaryotic cell or a eukaryotic cell. In an embodiment, the target sequence may be a non-natural DNA or RNA sequence.

In an embodiment, the target sequence may be present in a cell. In an embodiment, the target sequence may be present in the nucleus or cytoplasm (such as an organelle). In an embodiment, the cell may be a eukaryotic cell. In other embodiments, the cell may be a prokaryotic cell.

In an embodiment, the Cas protein may be linked to one or more NLS sequences. In an embodiment, the fusion protein may include one or more NLS sequences. In an embodiment, the NLS sequence may be linked to the N-terminus or C-terminus of the protein. In an embodiment, the NLS sequence may be fused to the N-terminus or C-terminus of the protein.

In another aspect, the present disclosure relates to an engineered CRISPR system, including the Cas protein and one or more gRNAs, where the gRNA includes a direct repeat and a spacer capable of hybridizing with a target nucleic acid, and the Cas protein can bind to the gRNA and target the target nucleic acid complementary to the spacer.

Protein-Nucleic Acid Complex/Composition

In another aspect, the present disclosure provides a complex/composition, including:

(i) a protein component selected from the group consisting of the Cas protein, the derivatized protein, the fusion protein, and any combination thereof; and

(ii) a nucleic acid component including: (a) a guide sequence capable of hybridizing with a target sequence and (b) a direct repeat capable of binding to the Cas protein of the present disclosure.

where the protein component and the nucleic acid component combine with each other to form a complex.

In an embodiment, the nucleic acid component may be a gRNA in the CRISPR-Cas system.

In an embodiment, the complex or composition may be non-naturally occurring or modified. In an embodiment, at least one component in the complex or composition may be non-naturally occurring or modified. In an embodiment, the first component may be non-naturally occurring or modified; and/or, the second component may be non-naturally occurring or modified.

Activated CRISPR Complex

In another aspect, the present disclosure also provides an activated CRISPR complex, including: (1) a protein component selected from the group consisting of the Cas protein, the derivatized protein, and the fusion protein of the present disclosure, and any combination thereof; (2) gRNA including: (a) a guide sequence capable of hybridizing with a target sequence and (b) a direct repeat capable of binding to the Cas protein of the present disclosure; and (3) a target sequence binding to the gRNA. Preferably, the binding may refer to binding between a nucleic acid-targeted targeting sequence on gRNA and a target nucleic acid.

The term “activated CRISPR complex”, “activated complex”, or “ternary complex” as used herein refers to a complex obtained after the Cas protein and gRNA in the CRISPR system bind to or are modified by a target nucleic acid.

The Cas protein and gRNA of the present disclosure can form a binary complex that is activated when binding to a nucleic acid substrate to form an activated CRISPR complex, where the nucleic acid substrate is complementary to a spacer (or called a guide sequence to hybridize with the target nucleic acid) in the gRNA. In some embodiments, the spacer of the gRNA may exactly match the target substrate. In other embodiments, the spacer of the gRNA may match a portion (continuous or discontinuous) of the target substrate.

In a preferred embodiment, the activated CRISPR complex may exhibit nuclease cleavage activity to the collateral nucleic acid, which refers to non-specific cleavage activity or disordered cleavage activity (which is also called trans cleavage activity in the art) of the activated CRISPR complex on a single-stranded nucleic acid.

Delivery and Delivery Composition

The Cas protein, gRNA, fusion protein, nucleic acid, vector, system, complex, and composition of the present disclosure can be delivered by any method known in the art. Such a method includes, but is not limited to, electroporation, lipofection, nucleofection, microinjection, sonoporation, gene gun, calcium phosphate-mediated transfection, cationic lipid transfection, lipofectin transfection, dendritic transfection, heat-shock transfection, magnetofection, puncture transfection, optical transfection, reagent-enhanced nucleic acid intake, and delivery via a liposome, an immunoliposome, a viral particle, an artificial virus, or the like.

Therefore, in another aspect, the present disclosure provides a delivery composition, which includes a delivery vehicle and one or more selected from the group consisting of the Cas protein, fusion protein, nucleic acid, vector, system, complex, and composition of the present disclosure.

In an embodiment, the delivery vehicle may be a particle.

In an embodiment, the delivery vehicle may be selected from the group consisting of a lipid particle, a sugar particle, a metal particle, a protein particle, a liposome, an exosome, a microvesicle, a gene guns, and a viral vector (such as replication-defective retrovirus, lentivirus, adenovirus, or AAV).

Host Cell

The present disclosure also relates to a cell or cell line or progeny thereof in vitro or in vivo, and the cell or cell line or progeny thereof includes the Cas protein, the fusion protein, the nucleic acid, the protein-nucleic acid complex, the activated CRISPR complex, the vector, or the delivery compositions of the present disclosure.

In some embodiments, the cell may be a prokaryotic cell.

In some embodiments, the cell may be a eukaryotic cell. In some embodiments, the cell may be a mammalian cell. In some embodiments, the cell may be a human cell. In some embodiments, the cell may be a non-human mammalian cell, such as a cell of a non-human primate, cow, sheep, pig, dog, monkey, rabbit, or a rodent (such as rat or mouse). In some embodiments, the cell may be a non-mammalian eukaryotic cell, such as a cell of a poultry bird (such as chicken), fish, or crustacea (such as clam or shrimp). In some embodiments, the cell may be a plant cell, such as a cell possessed by a monocotyledonous plant or a dicotyledonous plant or a cell possessed by a cultivated plant or a food crop such as cassava, corn, sorghum, soybean, wheat, oats, or rice. For example, the cell may be a cell possessed by algae, a tree, a production plant, a fruit, or a vegetable (for example, a tree such as a citrus tree or a nut tree; or a nightshade, cotton, tobacco, tomato, grape, coffee, cocoa, or the like).

In some embodiments, the cell may be a stem cell or a stem cell line.

In some cases, the host cell of the present disclosure may include a gene or a genome modification that is not present in the wild-type (WT) of the host cell.

Gene Editing Method and Use

The Cas protein, the nucleic acid, the composition, the CIRSPR/Cas system, the vector system, the delivery composition, the activated CRISPR complex, or the host cell of the present disclosure can be used in one or more selected from the group consisting of targeting and/or editing a target nucleic acid; cleaving a double-stranded DNA, a single-stranded DNA, or a single-stranded RNA; non-specifically cleaving and/or degrading a collateral nucleic acid; non-specifically cleaving a single-stranded nucleic acid; nucleic acid detection; detecting a nucleic acid in a target sample; specifically editing a double-stranded nucleic acid; base-editing a double-stranded nucleic acid; and base-editing a single-stranded nucleic acid. In other embodiments, the products of the present disclosure can also be used to prepare a reagent or a kit for one or more of the above purposes.

The present disclosure also provides use of the Cas protein, the nucleic acid, the composition, the CIRSPR/Cas system, the vector system, the delivery composition, or the activated CRISPR complex in gene editing, gene targeting, or gene cleaving; or use thereof in the preparation of a reagent or kit for gene editing, gene targeting, or gene cleaving.

In an embodiment, the gene editing, gene targeting, or gene cleaving may refer to gene editing, gene targeting, or gene cleaving inside and/or outside a cell.

The present disclosure also provides a method for editing, targeting, or cleaving a target nucleic acid, including: contacting the target nucleic acid with the Cas protein, the nucleic acid, the composition, the CIRSPR/Cas system, the vector system, the delivery composition, or the activated CRISPR complex. In an embodiment, the method may include the following: editing, targeting, or cleaving the target nucleic acid inside or outside a cell.

The gene editing or the editing a target nucleic acid may include modifying a gene, knocking out a gene, changing the expression of a gene product, repairing a mutation, and/or inserting a polynucleotide or a gene mutation.

The editing can be conducted in a prokaryotic cell and/or a eukaryotic cell.

In another aspect, the present disclosure also provides use of the Cas protein, the nucleic acid, the composition, the CIRSPR/Cas system, the vector system, the delivery composition, or the activated CRISPR complex in nucleic acid detection; or use thereof in the preparation of a reagent or kit for nucleic acid detection.

In another aspect, the present disclosure also provides a method for cleaving a single-stranded nucleic acid, including contacting a nucleic acid group with the Cas protein and the gRNA described above, where the nucleic acid group includes a target nucleic acid and a plurality of non-target single-stranded nucleic acids, and the Cas protein cleaves the plurality of non-target single-stranded nucleic acids.

The gRNA can bind to the Cas protein.

The gRNA can target the target nucleic acid.

The contacting can be conducted in vitro, in vivo or inside a cell.

Preferably, the cleavage of the single-stranded nucleic acid may refer to non-specific cleavage.

In another aspect, the present disclosure also provides use of the Cas protein, the nucleic acid, the composition, the CIRSPR/Cas system, the vector system, the delivery composition, or the activated CRISPR complex in the non-specific cleavage of a single-stranded nucleic acid; or use thereof in the preparation of a reagent or kit for the non-specific cleavage of a single-stranded nucleic acid.

In another aspect, the present disclosure also provides a kit for gene editing, gene targeting, or gene cleaving, including the Cas protein, the gRNA, the nucleic acid, the composition, the CIRSPR/Cas system, the vector system, the delivery composition, the activated CRISPR complex, or the host cell.

In another aspect, the present disclosure also provides a kit for detecting a target nucleic acid in a sample, including: (a) the Cas protein or a nucleic acid encoding the Cas protein; (b) the gRNA, or a nucleic acid encoding the gRNA, or a precursor RNA including the gRNA, or a nucleic acid encoding the precursor RNA; and (c) a single-stranded nucleic acid detector that does not hybridize with the gRNA.

It is known in the art that the precursor RNA can be cleaved or processed into the above-mentioned mature gRNA.

In another aspect, the present disclosure provides use of the Cas protein, the nucleic acid, the composition, the CIRSPR/Cas system, the vector system, the delivery composition, the activated CRISPR complex, or the host cell in the preparation of a formulation or a kit, where the preparation or the kit is used for:

(i) gene or genome editing;

(ii) target nucleic acid detection and/or diagnosis;

(iii) editing a target sequence in a target gene locus to modify an organism or a non-human organism;

(iv) disease treatment; and

(iv) targeting a target gene.

Preferably, the gene or genome editing may be conducted inside or outside a cell.

Preferably, the target nucleic acid detection and/or diagnosis may refer to target nucleic acid detection and/or diagnosis in vitro.

Preferably, the disease treatment may refer to treatment of a disease caused by a defect of a target sequence in a target gene locus.

In another aspect, the present disclosure provides a method for detecting a target nucleic acid in a sample, including: contacting the sample with the Cas protein, a gRNA, and a single-stranded nucleic acid detector; and detecting a detectable signal generated due to cleavage of the Cas protein on the single-stranded nucleic acid detector to detect the target nucleic acid; where the gRNA includes a region to bind to the Cas protein and a guide sequence to hybridize with a target nucleic acid, and the single-stranded nucleic acid detector does not hybridize with the gRNA.

Method for Specifically Modifying a Target Nucleic Acid

In another aspect, the present disclosure also provides a method for specifically modifying a target nucleic acid, including: contacting the target nucleic acid with the Cas protein, the nucleic acid, the composition, the CIRSPR/Cas system, the vector system, the delivery composition, or the activated CRISPR complex.

This specific modification may occur in vivo or in vitro.

This specific modification may occur inside or outside a cell.

In some cases, the cell may be selected from the group consisting of a prokaryotic cell and a eukaryotic cell, such as an animal cell, a plant cell, or a microbial cell.

In an embodiment, the modification may refer to a break in the target sequence, such as a single-strand break (SSB)/DSB in DNA or an SSB in RNA.

In some cases, the method may further include contacting the target nucleic acid with a donor polynucleotide, where the donor polynucleotide, a portion of the donor polynucleotide, a copy of the donor polynucleotide, or a portion of the copy of the donor polynucleotide is integrated into the target nucleic acid.

In an embodiment, the modification may further include inserting an edit template (such as an exogenous nucleic acid) into the break.

In an embodiment, the method may further include contacting an edit template with the target nucleic acid or delivering the edit template to a cell with the target nucleic acid. In an embodiment, the method may repair the broken target gene through homologous recombination with an exogenous template polynucleotide. In some embodiments, the repair may result in a mutation, including insertion, deletion, or substitution of one or more nucleotides in the target gene. In other embodiments, the mutation may result in one or more amino acid changes in a protein expressed by a gene carrying the target sequence.

Detection (Non-Specific Cleavage)

In another aspect, the present disclosure provides a method for detecting a target nucleic acid in a sample, including: contacting the sample with the Cas protein, the nucleic acid, the composition, the CIRSPR/Cas system, the vector system, the delivery composition, or the activated CRISPR complex and the single-stranded nucleic acid detector; and detecting a detectable signal generated due to cleavage of the Cas protein on the single-stranded nucleic acid detector to detect the target nucleic acid.

In the present disclosure, the target nucleic acid may include ribonucleotides or deoxyribonucleotides; and the target nucleic acid may include a single-stranded nucleic acid and a double-stranded nucleic acid, such as single-stranded DNA, double-stranded DNA, single-stranded RNA, and double-stranded RNA.

In an embodiment, the target nucleic acid may be derived from a sample such as a virus, a bacterium, a microorganism, soil, a water source, a human body, an animal, and a plant. Preferably, the target nucleic acid may be a product of enrichment or amplification by a method such as PCR, NASBA, RPA, SDA, LAMP, HAD, NEAR, MDA, RCA, LCR, or RAM.

In the present disclosure, the gRNA and the target sequence on the target nucleic acid may have a matching degree of at least 50%, preferably at least 60%, preferably at least 70%, preferably at least 80%, and preferably at least 90%.

In an embodiment, when the target sequence includes one or more characteristic sites (such as specific mutation sites or SNPs), the characteristic sites completely match the gRNA.

In an embodiment, the detection method may include one or more gRNAs with different guide sequences, which target different target sequences.

In the present disclosure, the single-stranded nucleic acid detector includes, but is not limited to, a single-stranded DNA, a single-stranded RNA, a DNA-RNA hybrid, a nucleic acid analogue, a base modifier, and a single-stranded nucleic acid detector with an abasic spacer; and the nucleic acid analogue includes, but is not limited to, locked nucleic acid (LNA), bridged nucleic acid (BNA), morpholino, glycol nucleic acid (GNA), hexitol nucleic acid (HNA), threose nucleic acid (TNA), arabinose nucleic acid (ANA), 2′-O-methyl RNA, 2′-methoxyacetyl RNA, 2′-fluoro RNA, 2′-amino RNA, 4′-thio RNA, and a combination thereof, including optional ribonucleotide or deoxyribonucleotide residues.

In the present disclosure, the detectable signal may be detected in the following ways: visual-based detection, sensor-based detection, color detection, fluorescence signal-based detection, gold nanoparticle-based detection, fluorescence polarization, colloidal phase transition/dispersion, electrochemical detection, and semiconductor-based detection.

In the present disclosure, preferably, two termini of the single-stranded nucleic acid detector may be provided with a fluorophore and a quencher respectively; and when the single-stranded nucleic acid detector is cleaved, a detectable fluorescence signal can be presented. The fluorophore may be one or more from the group consisting of FAM, FITC, VIC, JOE, TET, CY3, CYS, ROX, Texas Red, and LC RED460; and the quencher may be one or more from the group consisting of BHQ1, BHQ2, BHQ3, Dabcy1, and Tamra.

In other embodiments, a 5′ terminus and a 3′ terminus of the single-stranded nucleic acid detector may be provided with different labeling molecules respectively. The single-stranded nucleic acid detector is subjected to a colloidal gold test before and after being cleaved by the Cas protein; and the single-stranded nucleic acid detector shows different chromogenic results on the colloidal gold detection line and control line before and after being cleaved by the Cas protein.

In some embodiments, the method for detecting a target nucleic acid may further include: comparing a level of the detectable signal with a reference signal level, and determining an amount of the target nucleic acid in the sample based on the level of the detectable signal.

In some embodiments, the method for detecting a target nucleic acid may also include: using a RNA reporter nucleic acid and a DNA reporter nucleic acid (such as fluorescence color) on different channels, measuring a signal level of the RNA and DNA reporter molecules and an amount of the target nucleic acid in the RNA and DNA reporters to determine a level of the detectable signal, and sampling based on a combined (such as minimum or product) level of the detectable signal.

In an embodiment, the target gene may be present in a cell.

In an embodiment, the cell may be a prokaryotic cell.

In an embodiment, the cell may be a eukaryotic cell.

In an embodiment, the cell may be an animal cell.

In an embodiment, the cell may be a human cell.

In an embodiment, the cell may be a plant cell, such as a cell possessed by a cultivated plant (such as cassava, corn, sorghum, wheat, or rice), algae, a tree, or a vegetable.

In an embodiment, the target gene may be present in a nucleic acid in vitro (such as a plasmid).

In an embodiment, the target gene may be present in a plasmid.

### Terms and Definitions

In the present disclosure, unless otherwise specified, the scientific and technical terms used herein have the meanings commonly understood by those skilled in the art. In addition, the molecular genetics, nucleic acid chemistry, chemistry, molecular biology, biochemistry, cell culture, microbiology, cell biology, genomics, and recombinant DNA operation procedures used herein are routine procedures widely used in the corresponding fields. Moreover, in order to better explain the present disclosure, definitions and explanations of related terms are provided below.

Cas Protein

In the present disclosure, the terms “Cas protein”, “Cas enzyme”, and “Cas effector protein” can be used interchangeably. The inventors have discovered and identified a Cas effector protein for the first time, which has an amino acid sequence selected from the group consisting of the following sequences:

(i) a sequence shown in SEQ ID NO: 1;

(ii) a sequence obtained through substitution, deletion, or addition of one or more amino acids (such as substitution, deletion, or addition of 1, 2, 3, 4, 5, 6, 7, 8, 9, or 10 amino acids) based on the sequence shown in SEQ ID NO: 1; and

(iii) a sequence that has at least 20%, at least 30%, at least 40%, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% sequence identity with the sequence shown in SEQ ID NO: 1.

The nucleic acid cleavage or the cleavage of a nucleic acid herein may include: DNA or RNA break in a target nucleic acid caused by the Cas enzyme described herein (Cis cleavage), and DNA or RNA break in a collateral nucleic acid substrate (single-stranded nucleic acid substrate) (namely, non-specific or non-targeting Trans cleavage). In some embodiments, the cleavage may refer to a double-stranded DNA break. In some embodiments, the cleavage may refer to a single-stranded DNA break or a single-stranded RNA break.

CRISPR System

The terms “CRISPR-Cas system” and “CRISPR system” used herein can be used interchangeably and have the meaning commonly understood by those skilled in the art, which usually includes a transcription product or other elements related to the expression of a Cas gene, or a transcription product or other elements capable of guiding the activity of the Cas gene.

CRISPR/Cas Complex

As used herein, the term “CRISPR/Cas complex” refers to a complex formed by the binding of a gRNA or mature crRNA to the Cas protein, which includes a direct repeat that hybridizes with a guide sequence of the target sequence and binds to the Cas protein. The complex can recognize and cleave a polynucleotide capable of hybridizing with the gRNA or mature crRNA.

gRNA

As used herein, the terms “gRNA”, “mature crRNA”, and “guide sequence” can be used interchangeably and have the meaning commonly understood by those skilled in the art. Generally, a gRNA can include a direct repeat and a guide sequence, or is essentially composed of or is composed a direct repeat and a guide sequence.

In some cases, the guide sequence can be any polynucleotide sequence that shows sufficient complementarity with a target sequence to hybridize with the target sequence and guide the specific binding of the CRISPR/Cas complex to the target sequence. In an embodiment, under optimal alignment, a complementarity degree between the guide sequence and a corresponding target sequence may be at least 50%, at least 60%, at least 70%, at least 80%, at least 90%, at least 95%, or at least 99%. Determining the optimal alignment is within the competence of those of ordinary skill in the art. For example, there are published and commercially available alignment algorithms and programs, including but not limited to Smith-Waterman, Bowtie, Geneious, Biopython, and SeqMan in ClustalW and matlab.

Target Sequence

“Target sequence” refers to a polynucleotide targeted by a guide sequence in gRNA, such as a sequence that has complementarity with the guide sequence, where the hybridization between the target sequence and the guide sequence will promote the formation of a CRISPR/Cas complex (including Cas protein and gRNA). Complete complementarity is not necessary, as long as there is sufficient complementarity to cause hybridization and promote the formation of a CRISPR/Cas complex.

The target sequence can include any polynucleotide, such as DNA or RNA. In some cases, the target sequence may be located inside or outside a cell. In some cases, the target sequence may be located in the nucleus or cytoplasm of a cell. In some cases, the target sequence may be located in an organelle of a eukaryotic cell such as a mitochondrion or a chloroplast. A sequence or a template that can be recombined into a target gene locus with the target sequence is called “edit template”, “edit polynucleotide”, or “edit sequence”. In an embodiment, the edit template may be an exogenous nucleic acid. In an embodiment, the recombination may refer to homologous recombination.

In the present disclosure, the “target sequence”, “target polynucleotide”, or “target nucleic acid” can be any endogenous or exogenous polynucleotide for a cell (such as a eukaryotic cell). For example, the target polynucleotide may be a polynucleotide present in the nucleus of a eukaryotic cell. The target polynucleotide may be a sequence encoding a gene product (such as a protein) or a non-coding sequence (such as a regulatory polynucleotide or useless DNA). In some cases, the target sequence should be related to PAM.

Single-Stranded Nucleic Acid Detector

The single-stranded nucleic acid detector of the present disclosure refers to a sequence that includes 2 to 200 nucleotides, preferably 2 to 150 nucleotides, preferably 3 to 100 nucleotides, preferably 3 to 30 nucleotides, preferably 4 to 20 nucleotides, and preferably 5 to 15 nucleotides. Preferably, the single-stranded nucleic acid detector may be a single-stranded DNA molecule, a single-stranded RNA molecule, or a single-stranded DNA-RNA hybrid.

Two termini of the single-stranded nucleic acid detector include different reporter groups or labeling molecules. When the single-stranded nucleic acid detector is in an initial state (that is, when the single-stranded nucleic acid detector is not cleaved), no reporter signal is presented; and when the single-stranded nucleic acid detector is cleaved, a detectable signal is presented, indicating a detectable difference before and after cleavage.

In an embodiment, the reporter groups or labeling molecules may include fluorophores and quenchers. The fluorophores may be one or more from the group consisting of FAM, FITC, VIC, JOE, TET, CY3, CYS, ROX, Texas Red, and LC RED460; and the quenchers may be one or more from the group consisting of BHQ1, BHQ2, BHQ3, Dabcy1, and Tamra.

In an embodiment, the single-stranded nucleic acid detector may have a first molecule (such as FAM or FITC) linked to the 5′ terminus and a second molecule (such as biotin) linked to the 3′ terminus. The reaction system with a single-stranded nucleic acid detector may be used in combination with a flow strip to detect a target nucleic acid (preferably, colloidal gold detection). The flow strip is designed to have two capture lines, where an antibody to bind to a first molecule (namely, an anti-first molecule antibody) is arranged at a sample contact end (colloidal gold), an antibody to bind to the anti-first molecule antibody is arranged at a first line (control line), and an antibody to bind to a second molecule (namely, an anti-second molecule antibody, such as avidin) is arranged at a second line (test line). As a reaction proceeds along the strip, the anti-first molecule antibody binds to the first molecule and carries a cleaved or uncleaved oligonucleotide to the capture line, where a cleaved reporter will bind to the antibody binding to the anti-first molecule antibody at the first capture line; and an uncleaved reporter will bind to the anti-second molecule antibody at the second capture line. The binding of the reporter group to each line will result in a strong readout/signal (such as color). As more reporters are cut, more signals will accumulate at the first capture line, and fewer signals will appear at the second line. In some aspects, the present disclosure relates to use of the flow strip as described herein in the detection of a nucleic acid. In some aspects, the present disclosure relates to a method for detecting a nucleic acid using a flow strip as defined herein, such as a (lateral) flow test or a (lateral) flow immunochromatographic assay. In some aspects, the molecules in the single-stranded nucleic acid detector can be used instead of each other, or positions of the molecules can be changed. As long as a reporting principle is the same as or similar to that of the present disclosure, an improved method is also included in the present disclosure.

The detection method of the present disclosure can be used for quantitative detection of a target nucleic acid to be detected. The quantitative detection index can be quantified according to a signal intensity of a reporter group, for example, according to a luminous intensity of a fluorophore or according to a width of a chromogenic band.

Wild-Type

As used herein, the term “wild-type” has the meaning commonly understood by those skilled in the art, and indicates the typical form of an organism, a strain, or a gene, or a characteristic to distinguish the organism, strain, or gene in nature from a mutant or variant form thereof, which can be isolated from a natural source and is not artificially modified intentionally.

Derivatization

As used herein, the term “derivatization” refers to the chemical modification to an amino acid, a polypeptide, or a protein, where one or more substituents have been covalently linked to the amino acid, the polypeptide, or the protein. The substituents can also be referred to as side chains.

A derivatized protein is a derivative of a protein. Generally, the derivatization of a protein will not adversely affect the desired activity of the protein (for example, activity to bind to gRNA, endonuclease activity, and activity to bind to a specific site of a target sequence and cleave the target sequence under the guidance of gRNA). That is, a derivative of a protein has the same activity as the protein.

Derivatized Protein

A derivatized protein, also known as “protein derivative”, refers to a modified form of a protein, for example, one or more amino acids of the protein can be deleted, inserted, modified, and/or substituted.

Non-Natural

As used herein, the terms “non-natural” and “engineered” can be used interchangeably and refer to human intervention. When these terms are used to describe a nucleic acid or a polypeptide, it means that the nucleic acid or polypeptide is at least substantially isolated from a natural source or separated from at least another component binding to the nucleic acid or polypeptide in nature.

Orthologue

As used herein, the term “orthologue” has the meaning commonly understood by those skilled in the art. As a further guide, an orthologue of a protein described herein refers to a protein of a different species, which implements the same function as or the similar function to the protein.

Identity

As used herein, the term “identity” refers to the sequence matching between two polypeptides or between two nucleic acids. When specified positions in two sequences to be compared are occupied by the same base or amino acid monomer subunit (for example, a specified position in each of two DNA molecules is occupied by adenine, or a specified position in each of two peptides is occupied by lysine), the molecules are identical at the position. The “percentage identity” between two sequences is a function of the number of matched positions shared by the two sequences/the number of compared positions×100. For example, if 6 of 10 positions in a sequence match corresponding positions in another sequence, then the two sequences have 60% identity. For example, DNA sequences CTGACT and CAGGTT have 50% identity (3 of 6 positions are matching). Generally, the comparison is conducted when two sequences are aligned to produce maximum identity. Such alignment can be achieved by using, for example, a method of Needleman et al. (1970) J. Mol. Biol. 48: 443-453 that can be conveniently implemented by a computer program such as Align program (DNAstar, Inc.). The percentage identity between two amino acid sequences can also be determined by using an algorithm of E. Meyers and W. Miller (Comput. Appl Biosci., 4:11-17 (1988) integrated into the ALIGN program (version 2.0), a PAM120 weight residue table, a gap length penalty of 12, and a gap length penalty of 4. In addition, the percentage identity between two amino acid sequences can be determined by using Needleman and Wunsch (J Mol Biol. 48: 444-453 (1970)) algorithms in the GAP program integrated into the GCG software package (available on www.gcg.com), a Blossum 62 matrix or a PAM250 matrix, a gap weight of 16, 14, 12, 10, 8, 6, or 4, and a length weight of 1, 2, 3, 4, 5, or 6.

Vector

The term “vector” refers to a nucleic acid molecule that can deliver another nucleic acid linked thereto. The vector includes, but is not limited to, a single-stranded, double-stranded, or partially double-stranded nucleic acid; a nucleic acid with one or more free ends or without free ends (such as circular); DNA, RNA, or a nucleic acid of both; and other diverse polynucleotides known in the art. The vector can be introduced into a host cell through transformation, transduction, or transfection, such that a genetic material element carried by the vector can be expressed in the host cell. A vector can be introduced into a host cell to produce a transcript, a protein, or a peptide, including the protein, the fusion protein, the isolated nucleic acid, and the like (for example, a CRISPR transcript, such as a nucleic acid transcript, a protein, or an enzyme) described herein. A vector may include a variety of elements to control the expression, including but not limited to a promoter sequence, a transcription initiation sequence, an enhancer sequence, a selection element, and a reporter gene. In addition, the vector may also include a replication origin.

One type of vector is plasmid, which refers to a circular double-stranded DNA loop where an additional DNA fragment can be inserted, for example, by a standard molecular cloning technique.

Another type of vector is a viral vector, in which a virus-derived DNA or RNA sequence is present in a vector for packaging a virus (such as retrovirus, replication-defective retrovirus, adenovirus, replication-defective adenovirus, and AAV). A viral vector also includes a polynucleotide carried by a virus to be transfected into a host cell. Some vectors (for example, bacterial vectors with a bacterial replication origin and episomal mammalian vectors) can autonomously replicate in a host cell into which they are introduced.

Other vectors (such as non-episomal mammalian vectors) will be integrated into a genome of a host cell and thus replicate with the genome of the host cell after being introduced into the host cell. Moreover, some vectors can guide the expression of genes operably linked thereto. Such vectors are referred to as expression vectors herein.

Host Cell

As used herein, the term “host cell” refers to a cell that can be introduced with a vector, including, but not limited to, a prokaryotic cell such as Escherichia coli (E. coli) or Bacillus subtilis (B. subtilis), and a eukaryotic cell such as a fungal cell, an animal cell, and a plant cell, and a microbial cell.

Those skilled in the art will understand that the design of an expression vector may depend on factors such as the selection of a host cell to be transformed, and a desired expression level.

Regulatory Element

As used herein, the term “regulatory element” is intended to include a promoter, an enhancer, an IRES, and other expression control elements (for example, a transcriptional termination signal, such as a polyadenylation signal and a poly-U sequence), and the detailed description can be seen in Goeddel, “GENE EXPRESSION TECHNOLOGY: METHODS IN ENZYMOLOGY” 185, Academic Press, San Diego, Calif. (1990). In some cases, the regulatory element includes sequences that guide the constitutive expression of a nucleotide sequence in many types of host cells and sequences that guide the expression of the nucleotide sequence only in some host cells (such as a tissue-specific regulatory sequence). A tissue-specific promoter can mainly guide the expression in a desired tissue of interest, such as muscles, neurons, bones, skin, blood, specific organs (such as liver and pancreas), or specific cell types (such as lymphocytes). In some cases, a regulatory element can also guide the expression in a time-dependent manner (such as in a cell cycle-dependent or developmental stage-dependent manner), which may be or may not be tissue or cell type-specific. In some cases, the term “regulatory element” covers enhancer elements, such as WPRE; CMV enhancer; R-U5′ fragment in LTR of HTLV-I ((Mol. Cell. Biol., Vol 8 (1): 466-472, 1988); SV40 enhancer; and an intron sequence between exons 2 and 3 of rabbit β-globin (Proc. Natl. Acad. Sci. USA., Vol. 78 (3): 1527-31, 1981).

Promoter

As used herein, the term “promoter” has the meaning well known to those skilled in the art, which refers to a non-coding nucleotide sequence located upstream of a gene and capable of promoting the expression of a downstream gene. A constitutive promoter is a nucleotide sequence that will result in the generation of a gene product in a cell under most or all physiological conditions of the cell after the promoter is operably linked to a polynucleotide encoding or defining the gene product. An inducible promoter is a nucleotide sequence that will cause the generation of a gene product in a cell only when there is an inducer corresponding to the promoter in the cell after the promoter is operably linked to a polynucleotide encoding or defining the gene product. A tissue-specific promoter is a nucleotide sequence that will cause the generation of a gene product in a cell basically only when the cell is a cell of the tissue type corresponding to the promoter after the promoter is operably linked to a polynucleotide encoding or defining a gene product.

NLS

“NLS” (Nuclear Localization Signal) is an amino acid sequence that tags a protein for import into the nucleus through nuclear transport, that is, a protein with NLS is transported to the nucleus. Typically, NLS may include positively charged Lys or Arg residues that are exposed on the surface of a protein. Exemplary NLS includes, but is not limited to, SV40 large T antigen, EGL-13, c-Myc, and TUS protein. In some embodiments, the NLS may include a PKKKRKV sequence (SEQ ID NO: 11). In some embodiments, the NLS may include an AVKRPAATKKAGQAKKKKLD sequence (SEQ ID NO: 12). In some embodiments, the NLS may include a PAAKRVKLD sequence (SEQ ID NO: 13). In some embodiments, the NLS may include an MSRRRKANPTKLSENAKKLAKEVEN sequence (SEQ ID NO: 14). In some embodiments, the NLS may include a KLKIKRPVK sequence (SEQ ID NO: 15). Other NLS includes, but is not limited to, an acidic M9 domain of hnRNP A1, and sequences KIPIK (SEQ ID NO: 16) and PY-NLS in the yeast transcription repressor Mata2.

Operably Linked

As used herein, the term “operably linked” means that a nucleotide sequence of interest is linked to one or more regulatory elements in a manner that allows the expression of the nucleotide sequence (for example, in an in vitro transcription/translation system or in a host cell when the vector is introduced into the host cell).

Complementarity

As used herein, the term “complementarity” refers to the ability of a nucleic acid to form one or more hydrogen bonds with another nucleic acid by means of traditional Watson-Crick or another non-traditional form. The complementarity percentage refers to a percentage of residues in a first nucleic acid that can form hydrogen bonds (such as Watson-Crick base pairing) with a second nucleic acid (such as 5, 6, 7, 8, 9, and 10 of 10, namely 50%, 60%, 70%, 80%, 90%, and 100% complementarity). “Completely complementary” means that all consecutive residues of a first nucleic acid sequence form hydrogen bonds with the same number of consecutive residues in a second nucleic acid sequence. As used herein, “substantially complementary” means that there is at least 60%, 65%, 70%, 75%, 80%, 85%, 90%, 95%, 97%, 98%, 99%, or 100% complementarity in a region with 8, 9, 10, 11, 12, 13, 14, 15, 16, 17, 18, 19, 20, 21, 22, 23, 24, 25, 30, 35, 40, 45, 50, or more nucleotides, or means that two nucleic acids can hybridize under stringent conditions.

Stringent Conditions

As used herein, “stringent conditions” for hybridization refer to conditions under which a nucleic acid showing complementarity with a target sequence mainly hybridizes with the target sequence and substantially does not hybridize with a non-target sequence. Stringent conditions are usually sequence-dependent and vary depending on many factors. Generally, the longer the sequence, the higher the temperature at which the sequence specifically hybridizes with a corresponding target sequence.

Hybridization

The term “hybridization” or “complementary” or “substantially complementary” means that a nucleic acid (such as RNA and DNA) includes a nucleotide sequence that enables its non-covalent binding, that is, the nucleic acid can form base pairs and/or G/U base pairs with another nucleic acid in a sequence-specific, anti-parallel manner (namely, the nucleic acid specifically binds to a complementary nucleic acid), “annealing” or “hybridizing”.

The hybridization requires that two nucleic acids include complementary sequences. There may be mismatches between bases. Suitable conditions for hybridization between two nucleic acids depend on the length and complementarity degree of the nucleic acids, which are variables well known in the art. Typically, a hybridizable nucleic acid may include 8 nucleotides or more (such as 10 nucleotides or more, 12 nucleotides or more, 15 nucleotides or more, 20 nucleotides or more, 22 nucleotides or more, 25 nucleotides or more, or 30 nucleotides or more).

It should be understood that a sequence of a polynucleotide does not need to be 100% complementary to a sequence of its target nucleic acid for specific hybridization. A polynucleotide may have 60% or more, 65% or more, 70% or more, 75% or more, 80% or more, 85% or more, 90% or more, 95% or more, 98% or more, 99% or more, 99.5% or more, or 100% complementarity with a sequence of a target region in a target nucleic acid sequence to hybridize with the polynucleotide.

The hybridization of the target sequence with the gRNA means that the target sequence and the nucleic acid sequence of gRNA have at least 60%, 70%, 75%, 80%, 85%, 90%, 91%, 92%, 93%, 94%, 95%, 96%, 97%, 98%, 99%, or 100% complementarity, and thus can be hybridized to form a complex; or means that at least 12, 15, 16, 17, 18, 19, 20, 21, 22, or more bases in the target sequence are complementary to and paired with that in the nucleic acid sequence of gRNA, and thus the two sequences can be hybridized to form a complex.

Expression

As used herein, the term “expression” refers to a process by which a DNA template is transcribed into a polynucleotide (such as mRNA or another RNA transcript) and/or a process by which the transcribed mRNA is subsequently translated into a peptide, a polypeptide, or a protein. The transcript and the encoded polypeptide can be collectively referred to as “gene product”. If a polynucleotide is derived from genomic DNA (gDNA), the expression can include splicing of mRNA in a eukaryotic cell.

Linker

As used herein, the term “linker” refers to a linear polypeptide formed by linking a plurality of amino acid residues through peptide bonds. The linker of the present disclosure may be an artificially-synthesized amino acid sequence, or a natural polypeptide sequence, such as a polypeptide with a hinge domain function. Such linker polypeptides are well known in the art (see, for example, Holliger, P. et al. (1993) Proc. Natl. Acad. Sci. USA 90: 6444-6448; and Poljak, R. J. et al. (1994) Structure 2: 1121-1123).

Treatment

As used herein, the term “treatment” refers to treating or curing a disease, delaying the onset of symptoms of a disease, and/or delaying the development of a disease.

Subject

As used herein, the term “subject” includes, but is not limited to, various animals, plants, and microorganisms.

Animal

For example, the animal may be a mammal, such as bovine, equine, sheep, swine, canine, feline, leporid, rodent (such as mouse or rat), non-human primate (such as macaque or cynomolgus monkey), or human. In some embodiments, the subject (such as human) suffers from a disorder (such as a disorder caused by a disease-related gene defect).

Plant

The term “plant” should be understood as any differentiated multicellular organism capable of photosynthesis, including: crop plants at a mature or developmental stage, especially monocotyledonous or dicotyledonous plants; vegetable crops including artichoke, turnip cabbage, arugula, leek, asparagus, lettuce (such as head lettuce, leaf lettuce, and romaine lettuce), bok choy, malanga, melons (such as cantaloupe, watermelon, crenshaw melon, honeydew melon, and Roman cantaloupe), rape crops (such as Brussels sprout, cabbage, cauliflower, broccoli, borecole, kale, Chinese cabbage, and bok choy), cardoon, carrot, napa, okra, onion, celery, parsley, chickpea, parsnip, chicory, pepper, potato, gourd (such as marrow squash, cucumber, zucchini, cushaw, and pumpkin), radish, dried ball onion, rutabaga, purple eggplant (also known as eggplant), salsify, lettuce, shallot, endive, garlic, spinach, green onion, cushaw, greens, beets (sugar beets and fodder beets), sweet potato, Swiss chard, wasabi, tomato, turnip, and spices; fruits and/or vine crops such as apple, apricot, cherry, nectarine, peach, pear, plum, prune, quince, almond, chestnut, hazelnut, pecan, pistachio, walnut, citrus, blueberry, boysenberry, cranberry, currant, loganberry, raspberry, strawberry, blackberry, grape, avocado, banana, kiwi, persimmon, pomegranate, pineapple, tropical fruit, pome, melon, mango, papaya, and lychee; field crops, such as clover, alfalfa, evening primrose, meadowfoam, corn/maize (forage corn, sweet corn, and popcorn), lupulus, jojoba, peanut, rice, safflower, small grain crops (barley, oat, rye, wheat, and the like), sorghum, tobacco, kapok, legumes (beans, lentil, pea, and soybean), oil plants (canola, leaf mustard, poppy, olive, sunflower, coconut, castor oil plant, cocoa bean, and groundnut), Arabidopsis, fiber plants (cotton, flax, hemp, and jute), Lauraceae (cinnamon or camphor), or a plant such as coffee, sugar cane, tea, and natural rubber plants; and/or bedding plants such as a flowering plant, cactus, a succulent plant, and/or an ornamental plant, and trees such as forests (broad-leaved and evergreen trees, such as conifers), fruit trees, ornamental trees, nut-bearing trees, shrubs, and other seedlings.

### Advantageous Effects of the Present Disclosure

The present disclosure has discovered a novel Cas enzyme, which can exhibit nuclease activity in vivo and in vitro, and has promising application prospects.

Embodiments of the present disclosure will be described in detail below with reference to accompanying drawings and examples, but those skilled in the art will understand that the following accompanying drawings and examples are only used to illustrate the present disclosure rather than limit the scope of the present disclosure. Through the following detailed description of accompanying drawings and preferred embodiments, various objects and advantageous aspects of the present disclosure will become apparent to those skilled in the art.

### BRIEF DESCRIPTION OF THE DRAWINGS

FIG. 1 shows a PAM preference result of UkCpf1.

FIG. 2 shows a sterilization consumption experiment to verify the PAM preference result of UkCpf1.

FIG. 3 shows a functional domain prediction result of UkCpf1.

FIG. 4 shows in vitro RNA and DNA cleavage activity results of UkCpf1 and a mutant thereof.

FIG. 5 is a schematic diagram illustrating the construction of an UkCpf1 expression construct for Arabidopsis thaliana (A. thaliana).

FIG. 6 is a schematic diagram illustrating the principle of use of an YFFP gene (SEQ ID NO: 17) to detect UkCpf1 cleavage activity.

FIG. 7 shows the gene editing activity of UkCpf1 in A. thaliana cells.

FIG. 8 is a schematic diagram illustrating the construction of an UkCpf1 expression construct for rice.

FIG. 9 is a schematic diagram of the pDR-UkCpf1-At vector.

FIG. 10 shows a fluorescence result of nucleic acid detection of UkCpf1.

### SEQUENCE INFORMATION

| SEQ ID NO: | Description |
|---|---|
| 1 | Amino acid sequence of UkCpf1 |
| 2 | Nucleic acid sequence of UkCpf1 |
| 3 | DR region of gRNA of UkCpf1 |
| 4 | gTGW6-1 |
| 5 | gTGW6-2 |
| 6 | gTGW6-3 |
| 7 | gTGW6-4 |
| 8 | gTGW6-5 |
| 9 | N-B-i3g1-ssDNA0 |
| 10 | gRNA-trans |

### DETAILED DESCRIPTION OF THE EMBODIMENTS

The following examples are only used to describe rather than limit the present disclosure. Unless otherwise specified, the experiments and methods described in the examples are basically conducted in accordance with conventional methods well known in the art and described in various references. For example, conventional techniques such as immunology, biochemistry, chemistry, molecular biology, microbiology, cell biology, genomics, and recombinant DNA used in the present disclosure can be found in “MOLECULAR CLONING: A LABORATORY MANUAL”, Sambrook, Fritsch, and Maniatis, edition 2 (1989); “CURRENT PROTOCOLS IN MOLECULAR BIOLOGY” (edited by F. M. Ausubel et al., (1987)); and “METHODS IN ENZYMOLOGY” series (Academic Press Corporation): “PCR 2: A PRACTICAL APPROACH (edited by M. J. MacPherson, B. D. Hames, and G. R Taylor (1995)), ANTIBODIES, A LABORATORY MANUAL edited by Harlow and Lane (1988), and “ANIMAL CELL CULTURE” (edited by R. I. Freshney (1987)).

In addition, if no specific conditions are specified in the examples, the examples will be conducted according to conventional conditions or the conditions recommended by the manufacturer. All of the used reagents or instruments which are not specified with manufacturers are conventional commercially-available products. Those skilled in the art know that the present disclosure is described by way of examples in the embodiments, and the examples are not intended to limit the protection scope of the present disclosure. All publications and other references mentioned herein are incorporated into this article by reference in their entirety.

### Example 1. Acquisition of Cas Protein

The inventors analyzed the metagenome of an uncultivated microorganism and identified a novel Cas enzyme through de-redundancy and protein cluster analysis, and the novel Cas enzyme had an amino acid sequence shown in SEQ ID NO: 1 and a nucleic acid sequence shown in SEQ ID NO: 2. Blast results showed that the Cas protein had low sequence identity with reported Cas proteins; and the Cas protein was named UkCpf1 in the present disclosure.

Analysis results showed that a direct repeat of gRNA corresponding to the UkCpf1 protein was AUUUCUACUAUUGUAGAU (SEQ ID NO: 3), and corresponding PAM had a sequence shown in 5′-YYV-3′, where Y=C/T and V=C/G/A.

1.1 The PAM Preference of UkCpf1 was Tested Through a Bacterium Elimination Experiment.

In order to test the PAM site preference of UkCpf1, a UkCpf1 coding gene driven by a T7 promoter and a crRNA precursor driven by a J23119 promoter (namely, repeat-spacer-repeat DR-Sp-DR: TTGACAGCTAGCTCAGTCCTAGGTATAATACTAGTGTCTAAAGGTATTATAAAATTTCT ACTATTGTAGATAGAGCGCAATTAATTATTGCGGATATTCGTCTAAAGGTATTATAAAAT TTCTACTATTGTAGATTTTTTT, SEQ ID NO: 18) were ligated into a prokaryotic expression plasmid pET28a with kanamycin resistance, and then the prokaryotic expression plasmid was transformed into E. coli BL21 to obtain competent E. coli. Processed mature crRNA, namely gRNA, could identify a targeting site on a plasmid pACYCDuet with chloramphenicol resistance, and the targeting site included PAM composed of 8 random bases at the 5′-terminus and a recognition sequence with a length of 28 nt at the 3′-terminus. The PAM plasmid library was transformed into the above-mentioned competent E. coli, and then the E. coli was cultivated overnight at 37° C. Viable bacteria were collected the next day to extract the plasmid. The PAM site sequence of the obtained plasmid library was subjected to PCR amplification and sequenced, and an untransformed PAM library was used as a control group.

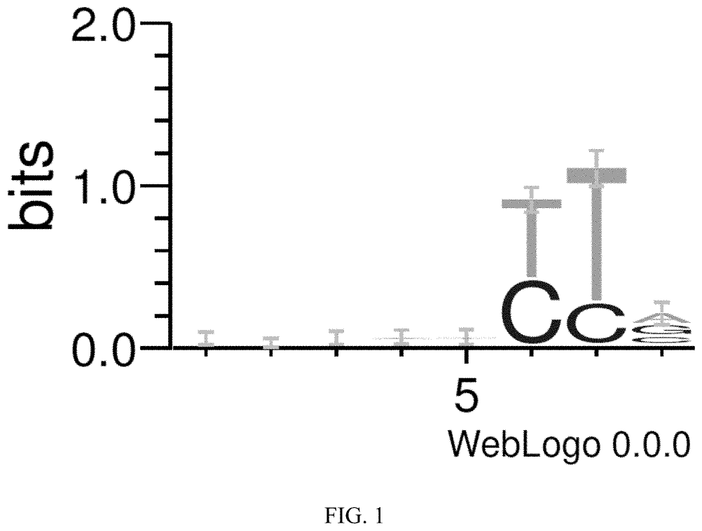

The abundance was counted for 65,536 PAM sequences in the experimental group and the control group, and data were standardized according to a sequencing depth. For any PAM sequence, when its log 2 (control group/sample group) was greater than 4.0, it was determined that the PAM was significantly consumed. A total of 825 significantly-consumed PAM sequences were obtained, accounting for 5.1% of all sequencing types. The Weblogo prediction of the 825 PAM sequences showed that the UkCpf1 preferred to cleave a target site with a 5′-terminus of a YYV (Y=C/T and V=C/G/A) sequence, and results were shown in FIG. 1. The preference was more relaxed and flexible than that of other known Cas12a (Cpf1) family members.

1.2 The PAM Preference of UkCpf1 was Verified Through a Sterilization Consumption Experiment.

In order to verify the PAM preference of UkCpf1 through a sterilization consumption experiment, a total of 32 PAM sequences with YYN were selected for bacteria test in vivo. Targeting sites that included the 32 PAMs and recognition sequences with a length of 28 nt were each linked to a pACYCDuet plasmid with chloramphenicol resistance, and then the plasmid was transformed into a competent E. coli strain expressing UkCpf1/gRNA. After a brief resuscitation at 37° C., concentrations of different transformed samples were leveled according to OD600 values of bacterial solutions, then the bacterial solutions were diluted to obtain three gradients: 10°, 10⁻¹, and 10⁻², and 5 μl of each bacterial solution was spotted on isopropyl-β-D-thiogalactoside (IPTG)-containing and IPTG-free chloramphenicol and kanamycin-resistant plates and cultivated overnight. The next day, colonies appearing on the plate were photographed and recorded.

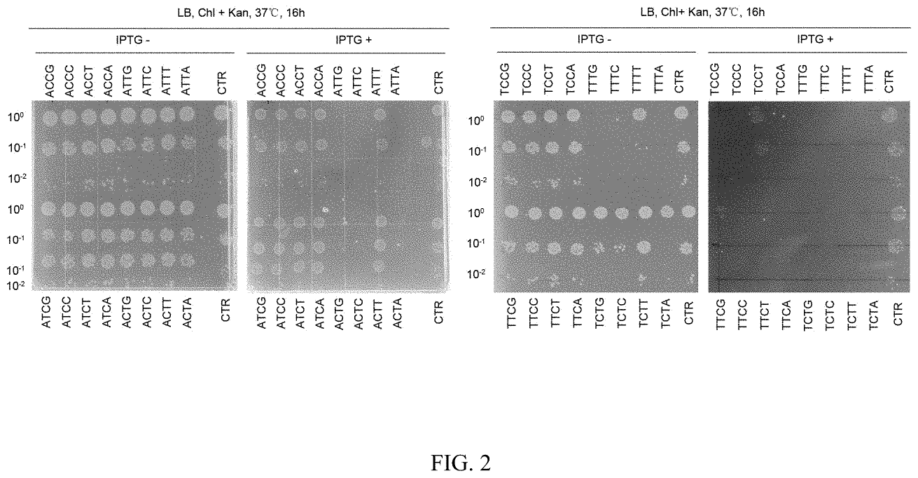

Results showed that the UkCpf1 only exhibited significant plasmid DNA cleavage activity for the “TTTV” type PAM on the IPTG-free plate. On the IPTG-containing plate, either “AYTV” or “TYYV” type PAM exhibited prominent cleavage activity. It indicated that the UkCpf1 preferentially recognized the “TYYV” type PAM site, and results were shown in FIG. 2.

1.3 Functional Domain and Catalytically-Active Site of UkCpf1

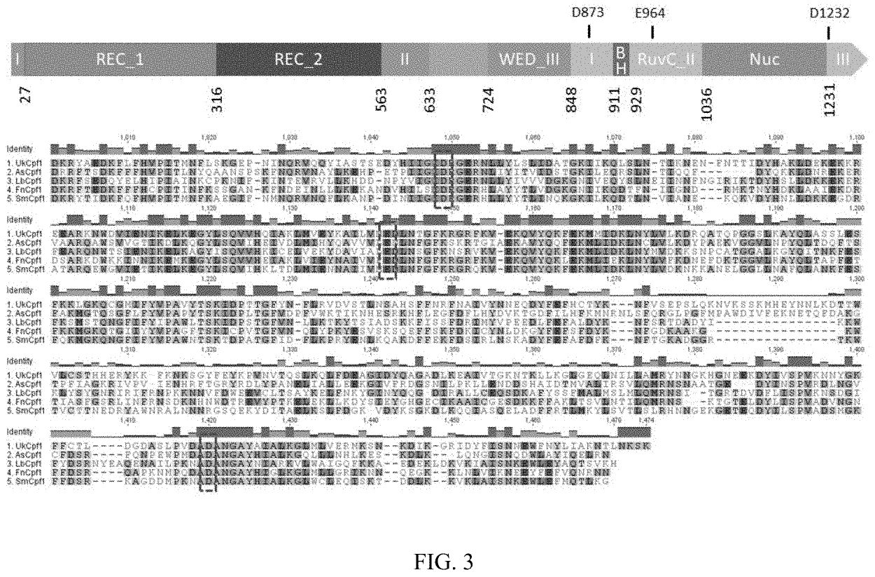

Amino acid sequences of UkCpf1 and four known Cpf1 were subjected to multiple sequence alignment with Muscle Alighment, and in combination with HHpred and HMM3_domain finder, a conservative domain of UkCpf1 was predicted. According to prediction results (shown in FIG. 3), three conservative catalytically-active sites of the RuvC domain were identified, including D873, E964, and D1232.

Coding sequences of FnCpf1 and LbCpf1 were synthesized and inserted into the pET28a plasmid for prokaryotic expression. D873, E964, and D1232 of UkCpf1 were mutated into D873A, E964A, and D1232A by overlap PCR respectively, then inserted into pET28a, and transformed into the E. coli strain BL21 together with a control plasmid of the wild-type UkCpf1, and positive clones were identified. Obtained positive clones were transferred to a test tube with 3 ml of a 100 mg/L kanamycin-containing LB medium, and cultivated overnight at 37° C. The next day, a resulting bacterial solution was inoculated at an inoculation ratio of 1:100 into a new Erlenmeyer flask with 20 ml of a 100 mg/L kanamycin-containing LB medium, and cultivated at 37° C. for about 8 h. In the afternoon of the next day, a resulting bacterial solution was inoculated at the inoculation ratio of 1:100 into a new Erlenmeyer flask with 1 L of a 100 mg/L kanamycin-containing LB medium, and cultivated at 37° C. until OD600 was 0.6 to 0.8. Then IPTG was added to a final concentration of 0.4 mM, and the bacteria were further cultivated for 18 h at 16° C. and 220 rpm. The bacteria were collected by centrifugation, and then passed through a nickel column, a heparin column, and a molecular sieve for purification to obtain the target protein.

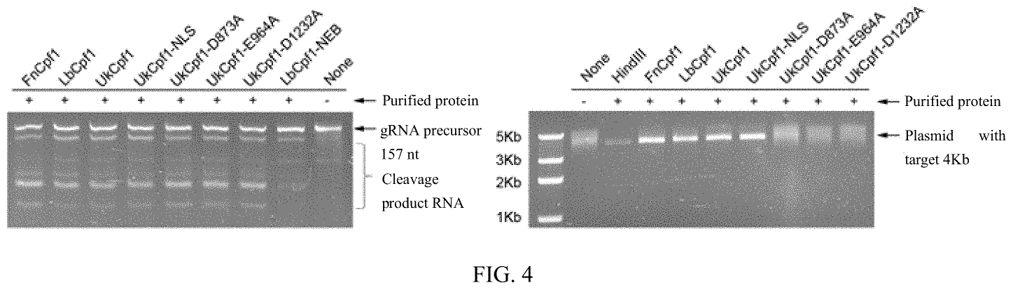

In order to determine whether UkCpf1 has the ability to process and cleave a precursor RNA, a precursor crRNA that had a length of 157 nt and included a sequence of DR-Sp-DR was transcribed in vitro. A reaction system was prepared by mixing 3 μl of 10×2.1 NEBbuffer, 2 μl of 10 μM Ukcpf1, 4 μl of 5 μM pre-crRNA, and 18 μl of DEPC H₂O, and then underwent a reaction at 25° C. for 30 min. Before RNA electrophoresis, a sample was digested with proteinase K at 25° C. for 15 min to remove Ukcpf1. A resulting reaction solution was loaded onto a 15% urea-PAGE gel to undergo electrophoresis for 2 h under tris-borate-EDTA (TBE) buffer, and then ethidium bromide (EB) staining and photographing were conducted. Results showed that UkCpf1 was similar to LbCpf1 and FnCpf1 and had the precursor RNA cleavage activity, and the mutations of D873A, E964A, and D1232A did not affect its RNA cleavage activity (see the left panel of FIG. 4).

In order to determine whether UkCpf1 has the cleavage activity against a target DNA, a pACYCDuet plasmid with the “TTTA” type PAM targeting site was constructed as a substrate to conduct a DNA cleavage experiment in vitro for identification. A reaction system was prepared by the same method as above, and then underwent a reaction at 25° C. for 30 min. 3 μl of a 100 ng/μl target plasmid was added to the reaction system, and then a reaction was conducted at 37° C. for 30 min. Digestion was conducted with proteinase K at 25° C. for 15 min, then a resulting reaction solution was loaded on a 0.8% agarose gel for TAE electrophoresis, and EB staining and photographing were conducted. Results showed that Ukcpf1 was similar to LbCpf1 and FnCpf1, which all could cleave a superspiral substrate DNA into a linear structure; and the predicted catalytically-active site mutation D873A, E964A, or D1232A of the RuvC domain caused Ukcpf1 to lose its DNA cleavage activity, indicating that these three sites are the catalytically-active sites of the RuvC domain (see the right panel of FIG. 4).

### Example 2. Editing Efficiency of UkCpf1 Protein in an A. thaliana Protoplast

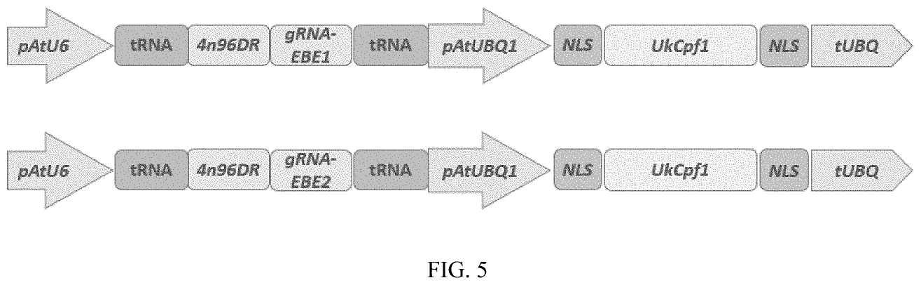

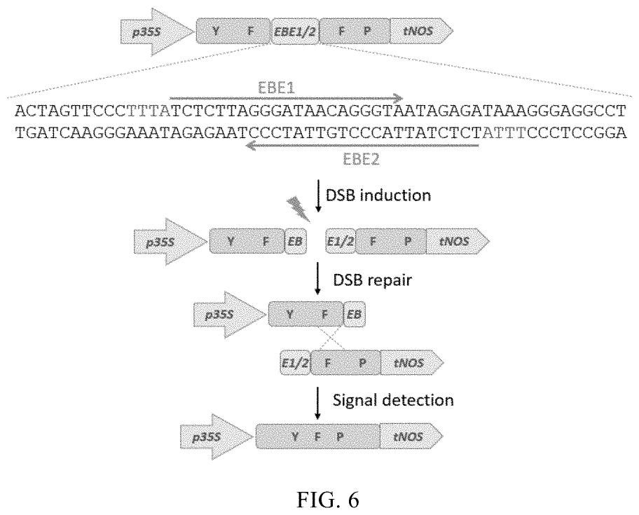

The engineered YFFP gene was used as a reporter to visualize the site-specific nuclease activity of UkCpf1 in an A. thaliana protoplast. Two UkCpf1 expression constructs were constructed to target EBE1 and EBE2 sites in a YFFP gene respectively. A schematic diagram of the constructs was shown in FIG. 5. Once cleaved by UkCpf1, a partially replicated “F” fragment will promote DSB repair through a homology-dependent DNA repair (HdR) pathway to restore the functional YFP gene (a schematic diagram was shown in FIG. 6). Therefore, the cleavage activity of UkCpf1 can be evaluated by observing the number of YFP-positive cells.

The isolation and preparation of A. thaliana protoplast cells were conducted according to the tape sandwich method reported in a literature. A reporter gene plasmid and a nuclease plasmid were mixed in a ratio of 1:1, and then transformed into protoplast cells by the PEG method. Transformed protoplast cells were cultivated in the dark at room temperature for 12 h to 24 h, then fluorescence signal channels of YFP and RFP were observed and photographed with a fluorescence stereo microscope (Olympus, IX71), and the number of YFP-positive cells was counted with ImageJ.

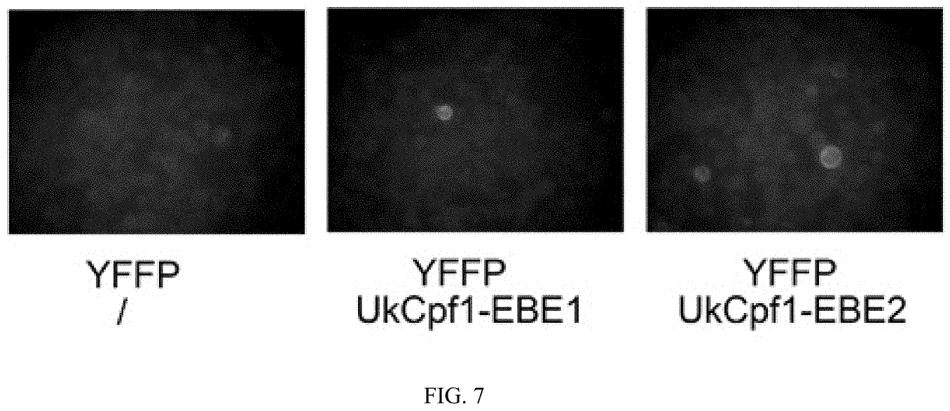

Results were shown in FIG. 7. Compared with the control, either for EBE1 or EBE2 site, the experimental group could show obvious fluorescent cells. That is, the UkCpf1 protein could show obvious cleavage activity in the A. thaliana protoplast and could be used for gene editing in cells.

### Example 3. Editing Efficiency of Cas Protein in a Rice Protoplast

With UkCpf1 in Example 1, the following 5 gRNAs were designed for a TGW6 gene of rice: gTGW6-1, gTGW6-2, gTGW6-3, gTGW6-4, and gTGW6-5. Targeting sequences of the above five gRNAs were: ACTACAAAACCGGCAACCTGTAC (SEQ ID NO: 4), TTTCACCGACAGCAGCATGAACT (SEQ ID NO: 5), TTGACCTGCCAGGCTATCCTGAT (SEQ ID NO: 6), GGTCCGGATAGTCACTTGGTTGC (SEQ ID NO: 7), and CGTGTAGCTGGGGCTGTACGTGT (SEQ ID NO: 8), respectively.

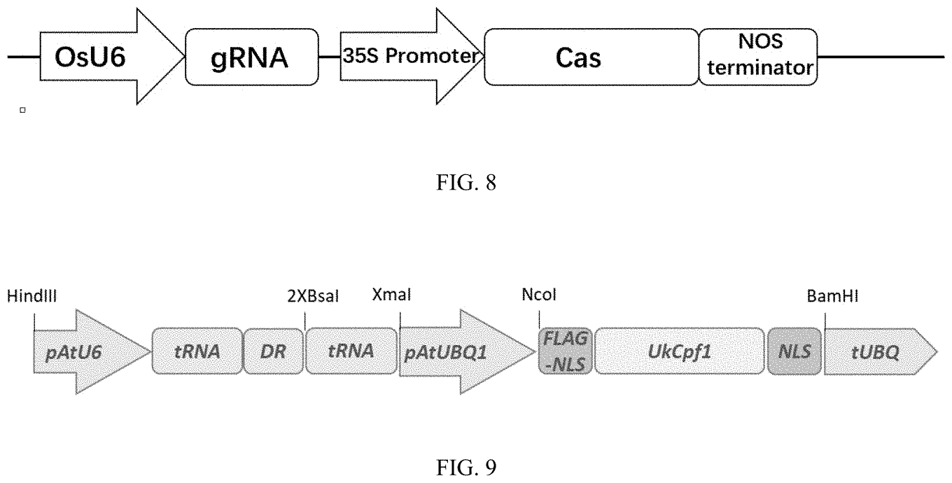

These 5 gRNAs were used to construct knockout vectors (as shown in FIG. 8), plasmids were extracted using the knockout vectors and transformed into corn protoplast cells, and the protoplast cells were cultivated in the dark at 37° C. for 24 h. After the cultivation was completed, a protoplast was collected by centrifugation, then protoplast DNA was extracted, and a DNA fragment of about 800 bp upstream and downstream of a target site was amplified. A DNA fragment with the target site was subjected to next-generation sequencing (NGS), and corresponding editing efficiency was counted; and the DNA fragment was compared with other Cas proteins, and results were shown in Table 2. The UkCpf1 protein of the present disclosure showed more efficient cleavage activity than other proteins in the rice protoplast.

TABLE 2
Editing efficiency of different Cas proteins in the rice protoplast

| | | Mapped | InDel | InDel |
|---|---|---|---|---|
| SampleID | AmpliconID | Reads | Reads | Reads Ratio |
| Cas160 | TGW6-1 | 878894 | 0 | 0.00% |
| | TGW6-2 | 2279912 | 2747 | 0.12% |
| | TGW6-3 | 1361224 | 0 | 0.00% |
| | TGW6-4 | 97 | 0 | 0.00% |
| | TGW6-5 | 1 | 0 | 0.00% |
| Cas230 | TGW6-1 | 1708137 | 0 | 0.00% |
| | TGW6-2 | 957129 | 867 | 0.09% |
| | TGW6-3 | 571055 | 0 | 0.00% |
| | TGW6-4 | 640298 | 98 | 0.02% |
| ukCpf1 | TGW6-1 | 1179912 | 177 | 0.02% |
| | TGW6-2 | 1975217 | 7672 | 0.39% |
| | TGW6-3 | 131813 | 748 | 0.57% |
| | TGW6-4 | 168485 | 528 | 0.31% |
| | TGW6-5 | 13431 | 98 | 0.73% |

### Example 4. Editing Efficiency of Cas Protein in A. thaliana

An A. thaliana material was selected from the Columbia wild-type background. Plant genetic transformation was conducted by the Agrobacterium GV3101-mediated floral dip method. Harvested T1-generation seeds were disinfected with 5% sodium hypochlorite for 10 min, rinsed 4 times with sterile water, and sown on a 30 μM hygromycin-resistant plate for screening. The plate was placed at 4° C. for 2 d and then incubated in a 12 h-light incubator for 10 d, and then resistant plants were transplanted into flower pots and further cultivated in a 16 h-light greenhouse.

The synthesized UkCpf1 sequence of Example 1 was amplified with primers pAtUBQ-F-UnCpf1/UnCpf1-R-tUBQ, and recombined to the NcoI and BamHI sites of the psgR-Cas9-At vector to obtain an intermediate vector psgR-UkCpf1-At. Then, the synthesized DR-tRNA site was ligated to the HindIII and XmaI sites of the psgR-UkCpf1-At vector through enzyme digestion to obtain a pDR-UkCpf1-At vector. A schematic diagram of the pDR-UkCpf1-At vector was shown in FIG. 9. The vector could be inserted into a target-specific sequence after undergoing BsaI digestion.

According to Table 3, sense and antisense primers targeting TT4-269 were synthesized. 10 plVI primers were denatured, annealed, and diluted (1/20), and then ligated to the 2×BsaI site of pDR-UkCpf1-At. A resulting vector could be transformed into Agrobacterium for genetic transformation of A. thaliana.

TABLE 3
Primers for pDR-UkCpf1-At vector construction

| | | SEQ ID |
|---|---|---|
| Primer | Sequence (5′-3′) | NO: |
| pAtUBQ-F- | GAGAGAGACGAAACACAAACCATGGAC | 19 |
| UnCpf1 | TACAAGGACCACGACGG | |
| UnCpf1-R-tUBQ | TTCTTGATAAGAGTCTCTTAGGATCCT | 20 |
| | CACTCCACCTTGCGCTTCTTCTTG | |
| AsDR-EBE-S1 | AGATTCTCTTAGGGATAACAGGGTAAT | 21 |
| AsDR-EBE-A1T | AAAAATTACCCTGTTATCCCTAAGAGA | 22 |
| AsDR-EBE-S2 | AGATTCTCTATTACCCTGTTATCCCTA | 23 |
| ASDR-EBE-A2T | AAAATAGGGATAACAGGGTAATAGAGA | 24 |
| AsDR-TT4-S269 | AGATCTATTCACAGGCGACAAGTCGAC | 25 |
| AsDR-TT4- | AAAAGTCGACTTGTCGCCTGTGAATAG | 26 |
| A269T | | |

For the A. thaliana transgenic T1-generation population of TT4-269, 52 lines were randomly selected, and one leaf was selected after each line grew for 2 weeks to extract the DNA genome by the cetyltrimethylammonium bromide (CTAB) method. A target gene fragment was amplified by PCR, and amplification products were used to build a library by the Hi-Tom method and sent to the Hiseq2500 platform for sequencing. For the data obtained, a linker sequence was cut off, and the remaining sequence was aligned with a reference gene sequence by bowtie. Alignment results were sorted by Samtool, and R was used for statistical mapping.

Final results showed that UkCpf1 exhibited significant editing effects in A. thaliana; for the TT4-269 target, in the 52 strains, the editing efficiency was as high as 65.4%; and the editing type mainly included single-base insertion and deletion. Another Cas protein SmCsm1 was used for editing at the above-mentioned site in A. thaliana, and results showed that its editing efficiency was only about 10%.

### Example 5. Use of Cas Protein in Nucleic Acid Detection

In this example, the trans cleavage activity of UkCpf1 was verified through an in vitro test. In this example, a gRNA that could be paired with a target nucleic acid was used to guide the UkCpf1 protein to recognize and bind to the target nucleic acid; then the trans cleavage activity of the UkCpf1 protein to the single-stranded nucleic acid was stimulated to cleave the single-stranded nucleic acid detector in the system; two termini of the single-stranded nucleic acid detector were provided with a fluorophore and a quencher respectively, and if the single-stranded nucleic acid detector was cleaved, fluorescence will be excited; and in other embodiments, the two termini of the single-stranded nucleic acid detector could also be provided with a labeling molecule that could be detected by colloidal gold.

In this example, a selected target nucleic acid was a single-stranded DNA, N-B-i3g1-ssDNA0, with a sequence:

(SEQ ID NO: 9)
CGACATTCCGAAGAACGCTGAAGCGCTGGGGGCAAATTGTGCAATTTGCG
GC;

a gRNA sequence was

(SEQ ID NO: 10)
AGAGAAUGUGUGCAUAGUCACACCCCCCAGCGCUUCAGCGUUC;

and

a sequence of a single-stranded nucleic acid detector was FAM-TTGTT-BHQ1.

The following reaction system was adopted: UkCpf1 with a final concentration of 50 nM, gRNA with a final concentration of 50 nM, target nucleic acid with a final concentration of 500 nM, and single-stranded nucleic acid detector with a final concentration of 200 nM. The reaction system was incubated at 37° C. and then the FAM fluorescence was read every 1 min. No target nucleic acid was added in the control group.

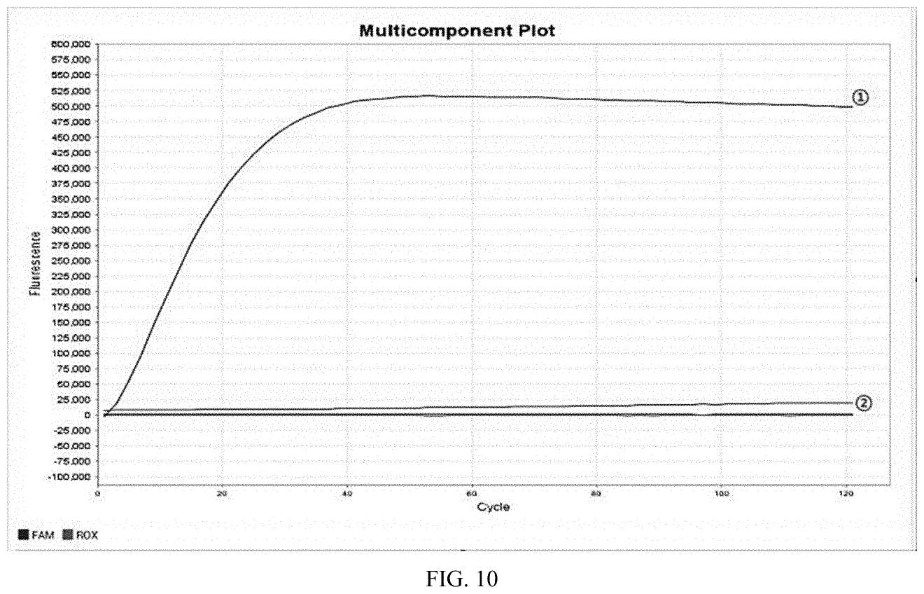

As shown in FIG. 10, compared with the target nucleic acid-free control, in the presence of target nucleic acid, single-stranded nucleic acid detection in the UkCpf1 cleavage system quickly reported fluorescence. The above experiment showed that, in combination with the single-stranded nucleic acid detector, UkCpf1 can be used for target nucleic acid detection. In FIG. 10, \textcircled{1} shows the experimental result of the group with the target nucleic acid, and \textcircled{2} shows the experimental result of the control group without the target nucleic acid.

### Example 6. UkCpf1-Mediated PDS Gene Mutations in A. thaliana and Rice

In order to determine whether UkCpf1 can edit a genome of a plant cell, a plant stable expression vector suitable for rice and A. thaliana was constructed. The UBI promoter (pZmUBI) and the RPS5a (pRPS5a) were used to drive the stable expression of the UKCpf1 gene in rice and A. thaliana respectively, and the rice U6 promoter (pU6) and the A. thaliana U6 promoter (pU6) were used to drive the expression of the crRNA element (DR-guide) of UKCpf1 in rice and A. thaliana respectively. In order to improve the accuracy and stability of expression of the 3′ terminus of the crRNA element in A. thaliana, the HDV ribozyme sequence was fusion-expressed at the 3′ terminus of crRNA. The PDS genes of rice and A. thaliana were each used as an identification target of crRNA to facilitate the calculation of gene editing efficiency through the phenotype of leaf bleaching.

The above-mentioned two vectors were introduced into the genomes of rice and A. thaliana respectively through Agrobacterium-mediated plant genetic transformation, and screening was conducted with hygromycin to obtain stably-transformed transgenic materials. Primers (AtPDS-F: 5′-GGTCCTTTGCAGGTATCT-3′, as shown in SEQ ID NO: 27, and AtPDS-R: 5′-TTCAAAGGCTTAGCAGGACGA-3′, as shown in SEQ ID NO: 28) were used to sequence and identify targets, and the leaf bleaching phenotypes of genetically-modified materials were counted. Results showed that the UkCpf1 had editing efficiency of 7% and 44% on PDS genes in rice and A. thaliana, respectively.

### Example 7. UkCpf1-Mediated DNMT1 Gene Editing in Human Cell Line 293T

In order to determine whether UkCpf1 can be used for gene editing in human cells, an UkCpf1 expression vector suitable for human cells was constructed. The CAG promoter (pCAG) was used to drive the expression of UkCpf1, and the human U6 promoter (pHuU6) was used to drive the chimeric sequences of crRNA and HDV ribozyme. In the human DNMT1 gene coding sequence, TTV, TCV, CTV, and CCV were selected as targeting sites for PAM. The resulting plasmid vector was introduced into human 293T cells by lipofectin transfection. After the cells were cultivated for 2 d, the gDNA was extracted from the cells, and a DNA sequence of a target site was subjected to PCR amplification and sequencing with primers (DNMT1-F: 5′-CGGGAACCAAGCAAGAAGTG-3′, as shown in SEQ ID NO: 29, and DNMT1-R: 5′-GGGCAACACAGTGAGACTCC-3′, as shown in SEQ ID NO: 30). According to statistical results of Sanger and high-throughput sequencing, UkCpf1 showed editing activity on these four targets, with the highest editing efficiency of 14.5%.

Although the specific implementations of the present disclosure have been described in detail, those skilled in the art will understand that various modifications and changes can be made to the details according to all teachings published, and such modifications and changes are all within the protection scope of the present disclosure. The full content of the present disclosure is defined by the appended claims and any equivalents thereof.

What is claimed is:

1. A clustered regularly interspaced short palindromic repeat (CRISPR)-associated (Cas) protein, wherein the Cas protein is any one from the group consisting of:
a first Cas protein having an amino acid sequence with at least 95% sequence identity with SEQ ID NO: 1 and basically retaining a biological function of SEQ ID NO: 1;
a second Cas protein having an amino acid sequence obtained through a substitution, a deletion, or an addition of one or more amino acids based on SEQ ID NO: 1 and basically retaining the biological function of SEQ ID NO: 1, and the one or more amino acids comprise 1, 2, 3, 4, 5, 6, 7, 8, 9, or 10 amino acids; and
a third Cas protein comprising an amino acid sequence shown in SEQ ID NO: 1.

2. A fusion protein, comprising the Cas protein according to claim 1 and a modification part.

3. An isolated polynucleotide, wherein the isolated polynucleotide is a polynucleotide sequence encoding the Cas protein according to claim 1, or a polynucleotide sequence encoding a fusion protein comprising the Cas protein and a modification part.

4. A guide RNA (gRNA), comprising a framework region binding to the Cas protein according to claim 1 and a guide sequence targeting a target sequence.

5. A vector, comprising the isolated polynucleotide according to claim 3 and a regulatory element operably linked to the isolated polynucleotide.

6. A CRISPR-Cas system, comprising the Cas protein according to claim 1 and at least one gRNA, wherein the at least one gRNA comprises a framework region binding to the Cas protein and a guide sequence targeting a target sequence.

7. A vector system, wherein the vector system comprises one or more vectors, and the one or more vectors comprise:
a) a first regulatory element operably linked to a gRNA, wherein the gRNA comprises a framework region binding to the Cas protein according to claim 1 and a guide sequence targeting a target sequence, and
b) a second regulatory element operably linked to the Cas protein;
wherein the first regulatory element and the second regulatory element are located on a same vector or different vectors of the vector system.

8. A composition, comprising:
a protein component selected from the group consisting of the Cas protein according to claim 1 and a fusion protein comprising the Cas protein and a modification part; and
a nucleic acid component selected from the group consisting of a gRNA comprising a framework region binding to the Cas protein and a guide sequence targeting a target sequence, a nucleic acid encoding the gRNA, a precursor RNA of the gRNA, and a nucleic acid encoding the precursor RNA of the gRNA;
wherein the protein component and the nucleic acid component combine with each other to form the composition.

9. An activated CRISPR complex, comprising:
a protein component selected from the group consisting of the Cas protein according to claim 1 and a fusion protein comprising the Cas protein and a modification part;
a nucleic acid component selected from the group consisting of a gRNA comprising a framework region binding to the Cas protein and a guide sequence targeting a target sequence, a nucleic acid encoding the gRNA, a precursor RNA of the gRNA, and a nucleic acid encoding the precursor RNA of the gRNA; and
the target sequence binding to the gRNA.

10. An engineered host cell, comprising:
the Cas protein according to claim 1, or
a fusion protein comprising the Cas protein and a modification part, or
a polynucleotide, wherein the polynucleotide is a polynucleotide sequence encoding the Cas protein or a polynucleotide sequence encoding the fusion protein, or
a vector, wherein the vector comprises the polynucleotide and a first regulatory element operably linked to the polynucleotide, or
a CRISPR-Cas system comprising the Cas protein and at least one gRNA, wherein the at least one gRNA comprises a framework region binding to the Cas protein and a guide sequence targeting a target sequence, or
a vector system, wherein the vector system comprises one or more vectors, and the one or more vectors comprise a second regulatory element operably linked to the gRNA, and a third regulatory element operably linked to the Cas protein, wherein the second regulatory element and the third regulatory element are located on a same vector or different vectors of the vector system, or
a composition, wherein the composition comprises a protein component selected from the group consisting of the Cas protein and the fusion protein; and a nucleic acid component selected from the group consisting of the gRNA, a nucleic acid encoding the gRNA, a precursor RNA of the gRNA, and a nucleic acid encoding the precursor RNA of the gRNA, wherein the protein component and the nucleic acid component combine with each other to form the composition, or
an activated CRISPR complex, wherein the activated CRISPR complex comprises the protein component; the nucleic acid component; and the target sequence binding to the gRNA.

11. The Cas protein according to claim 1, wherein the Cas protein is used in a gene editing, a gene targeting, or a gene cleaving.

12. The Cas protein according to claim 1, wherein the Cas protein is used in one or more selected from the group consisting of:
targeting and/or editing a target nucleic acid; cleaving a double-stranded DNA, a single-stranded DNA, or a single-stranded RNA; non-specifically cleaving and/or degrading a collateral nucleic acid; non-specifically cleaving a single-stranded nucleic acid; a nucleic acid detection; specifically editing a double-stranded nucleic acid; base-editing the double-stranded nucleic acid; and base-editing the single-stranded nucleic acid.

13. A method for editing a target nucleic acid, targeting the target nucleic acid, or cleaving the target nucleic acid, comprising: contacting the target nucleic acid with the Cas protein according to claim 1, or a fusion protein comprising the Cas protein and a modification part, or a polynucleotide, wherein the polynucleotide is a polynucleotide sequence encoding the Cas protein or a polynucleotide sequence encoding the fusion protein, or a vector, wherein the vector comprises the polynucleotide and a first regulatory element operably linked to the polynucleotide, or a CRISPR-Cas system comprising the Cas protein and at least one gRNA, wherein the at least one gRNA comprises a framework region binding to the Cas protein and a guide sequence targeting a target sequence, or a vector system, wherein the vector system comprises one or more vectors, and the one or more vectors comprise a second regulatory element operably linked to the gRNA, and a third regulatory element operably linked to the Cas protein, wherein the second regulatory element and the third regulatory element are located on a same vector or different vectors of the vector system, or a composition wherein the composition comprises a protein component selected from the group consisting of the Cas protein and the fusion protein; and a nucleic acid component selected from the group consisting of the gRNA, a nucleic acid encoding the gRNA, a precursor RNA of the gRNA, and a nucleic acid encoding the precursor RNA of the gRNA, wherein the protein component and the nucleic acid component combine with each other to form the composition, or an activated CRISPR complex, wherein the activated CRISPR complex comprises the protein component; the nucleic acid component; and the target sequence binding to the gRNA, or a host cell, wherein the host cell comprises the Cas protein, the fusion protein, the polynucleotide, the vector, the CRISPR-Cas system, the vector system, the composition, or the activated CRISPR complex.

14. A method for cleaving a single-stranded nucleic acid, comprising: contacting a nucleic acid group with the Cas protein according to claim 1 and a gRNA comprising a framework region binding to the Cas protein and a guide sequence targeting a target sequence, wherein the nucleic acid group comprises a target nucleic acid and at least one non-target single-stranded nucleic acid; the gRNA targets the target nucleic acid; and the Cas protein cleaves the non-target single-stranded nucleic acid.

15. A kit for gene editing, gene targeting, or gene cleaving, comprising:
the Cas protein according to claim 1, or
a fusion protein comprising the Cas protein and a modification part, or
a polynucleotide, wherein the polynucleotide is a polynucleotide sequence encoding the Cas protein or a polynucleotide sequence encoding the fusion protein, or
a vector, wherein the vector comprises the polynucleotide and a first regulatory element operably linked to the polynucleotide, or
a CRISPR-Cas system comprising the Cas protein and at least one gRNA, wherein the at least one gRNA comprises a framework region binding to the Cas protein and a guide sequence targeting a target sequence, or
a vector system, wherein the vector system comprises one or more vectors, and the one or more vectors comprise a second regulatory element operably linked to the gRNA, and a third regulatory element operably linked to the Cas protein, wherein the second regulatory element and the third regulatory element are located on a same vector or different vectors of the vector system, or
a composition, wherein the composition comprises a protein component selected from the group consisting of the Cas protein and the fusion protein; and a nucleic acid component selected from the group consisting of the gRNA, a nucleic acid encoding the gRNA, a precursor RNA of the gRNA, and a nucleic acid encoding the precursor RNA of the gRNA, wherein the protein component and the nucleic acid component combine with each other to form the composition, or
an activated CRISPR complex, wherein the activated CRISPR complex comprises the protein component; the nucleic acid component; and the target sequence binding to the gRNA, or
a host cell, wherein the host cell comprises the Cas protein, the fusion protein, the polynucleotide, the vector, the CRISPR-Cas system, the vector system, the composition, or the activated CRISPR complex.

16. A kit for detecting a target nucleic acid in a sample, comprising: (a) the Cas protein according to claim 1 or a nucleic acid encoding the Cas protein; (b) a gRNA comprising a framework region binding to the Cas protein and a guide sequence targeting a target sequence, or a nucleic acid encoding the gRNA, or a precursor RNA comprising the gRNA, or a nucleic acid encoding the precursor RNA; and (c) a single-stranded nucleic acid detector not hybridizing with the gRNA.

17. The Cas protein according to claim 1, wherein the Cas protein is used in a preparation of a formulation or a kit, wherein the formulation or the kit is used for:
(i) gene or genome editing;
(ii) target nucleic acid detection and/or diagnosis;
(iii) editing a target sequence in a target gene locus to modify an organism or a non-human organism;
(iv) disease treatment; and
(v) targeting the target gene.

18. A method for detecting a target nucleic acid in a sample, comprising: contacting the sample with the Cas protein according to claim 1, a gRNA, and a single-stranded nucleic acid detector; and detecting a detectable signal generated due to a cleavage of the Cas protein on the single-stranded nucleic acid detector to detect the target nucleic acid; wherein the gRNA comprises a region to bind to the Cas protein and a guide sequence to hybridize with the target nucleic acid, and the single-stranded nucleic acid detector does not hybridize with the gRNA.
